(19) United States
(12) Patent Application Publication
(10) Pub. No.: US 2023/0036583 A1
(43) Pub. Date: Feb. 2, 2023

(54) DEVICES AND METHODS FOR REDUCING REJECTION OF A TRANSPLANTED ORGAN IN A RECIPIENT

(71) Applicant: SeaStar Medical, Inc., Denver, CO (US)

(72) Inventors: James R. Matson, Westminster, CO (US); Christopher Jaynes, Centennial, CO (US); Charles Jack Fisher, JR., Cardiff-By-The-Sea, CA (US)

(21) Appl. No.: 17/789,955

(22) PCT Filed: Dec. 30, 2020
(86) PCT No.: PCT/US2020/067527

§ 371 Date: Jun. 29, 2022

Related U.S. Application Data
(60) Provisional application No. 62/955,635, filed on Dec. 31, 2019.

(51) Int. Cl. A61M 1/34; A61M 1/36; A01N 1/02

(52) U.S. Cl. CPC A61M 1/3401; A61M 1/362264; A01N 1/0247

(57) ABSTRACT

The invention relates generally to methods of improving function of a transplanted organ, treating or preventing primary graft dysfunction of a transplanted organ, treating or preventing acute rejection of a transplanted organ, treating or preventing delayed graft function, or achieving a clinical endpoint indicative of a successful organ transplant in a recipient of the transplanted organ which comprise contacting blood from the recipient with an extracorporeal membrane having a plurality of pores having an average pore size of at least 40 kDa, 50 kDa or 60 kDa to permit inflammatory cytokines and other inflammatory molecules to pass through the pores and out of the blood that is returned back to the recipient.

### CROSS-REFERENCE TO RELATED APPLICATIONS

This application claims priority to and the benefit of U.S. Provisional Patent Application No. 62/955,635 filed Dec. 31, 2019, the contents of which are incorporated by reference herein in their entirety.

### FIELD OF THE INVENTION

The invention involves treatment of blood by extracorporeal circulation in subjects receiving an organ transplant.

### BACKGROUND

While receiving a transplanted organ (such as a kidney, liver, heart, or lung) is the gold-standard treatment for a subject experiencing organ failure, transplanted organs are at risk of being rejected by the recipient's immune system. Acute rejection of a transplanted organ generally occurs from the time of transplantation up to 3 months afterward but can occur within several months up to a year after transplant, whereas chronic rejection takes place over many years during which the body's constant immune response against the transplanted organ slowly damages the organ and reduces the organ's function. Rejection is a serious complication of organ transplantation, resulting in decreased organ function, organ destruction, and ultimately death of a subject. Typically, a subject is closely matched with a donor based on phenotypic and genotypic similarities and receives immunosuppressant drugs to stave off rejection. However, some form of rejection still occurs in more than 25% of liver transplant recipients, in more than 30% of kidney transplant recipients, and in more than 50% of cardiac transplant recipients.

A donor organ in a brain-dead organ donor is exposed to toxic inflammation at a systemic level. Inflammation both directly damages the tissues of the vital organ, and activates resident immune cells. On engraftment, the resident immune cells in the graft organ interact with the recipient's immune system; this can drive early activation of the recipient's immune response driving earlier, more aggressive rejection. Additionally, when the donor organ is recovered from the donor, the organ's blood supply is interrupted which begins a variable period of ischemia. This ischemia causes further direct tissue injury in the graft organ, and exacerbates resident inflammation. The injured donor organ stimulates recipient inflammation which promotes further graft organ injury and increased risk of organ dysfunction, failure or rejection.

Accordingly, there is a need for treatments and methods to improve outcomes in organ transplant recipients including, for example, treating or preventing graft rejection and/or graft dysfunction.

### SUMMARY OF THE INVENTION

The invention is based, in part, upon the discovery that outcomes in a recipient of a transplanted organ can be improved by contacting blood from the recipient with a semi-permeable membrane, for example, one or more membranes disposed in a hemofilter, whereupon, inflammatory cytokines or other inflammatory molecules can pass through semi-permeable membrane. Blood depleted of inflammatory molecules is then returned to the subject. Without wishing to be bound by theory, it is contemplated that processing the recipient blood using this approach removes inflammatory cytokines and other inflammatory molecules from the blood that negatively impact organ function and cause inflammatory responses that ultimately lead to organ rejection.

Accordingly, in one aspect, the invention provides a method of improving a function of a transplanted organ in a recipient of the transplanted organ. In one embodiment, the method involves contacting blood from the recipient with an extracorporeal membrane. The extracorporeal membrane defines a plurality of pores having an average pore size of at least 40 kDa, 50 kDa or 60 kDa that permit inflammatory molecules in the blood to pass therethrough for removal from the blood. The blood depleted of inflammatory molecules is then returned to the recipient.

In another aspect, the invention provides a method of treating or preventing primary graft dysfunction of a transplanted organ in a recipient of the transplanted organ. In one embodiment, the method involves contacting blood from the recipient with an extracorporeal membrane. The extracorporeal membrane defines a plurality of pores having an average pore size of at least 40 kDa, 50 kDa or 60 kDa that permit inflammatory molecules in the blood to pass therethrough for removal from the blood. The blood depleted of inflammatory molecules is then returned to the recipient.

In another aspect, the invention provides a method of treating or preventing acute rejection of a transplanted organ in a recipient of the transplanted organ. In one embodiment, the method involves contacting blood from the recipient with an extracorporeal membrane. The extracorporeal membrane defines a plurality of pores having an average pore size of at least 40 kDa, 50 kDa or 60 kDa that permit inflammatory molecules in the blood to pass therethrough for removal from the blood. The blood depleted of inflammatory molecules is then returned to the recipient.

In another aspect, the invention provides a method of treating or preventing delayed graft function of a transplanted organ in a recipient of the transplanted organ. In one embodiment, the method involves contacting blood from the recipient with an extracorporeal membrane. The extracorporeal membrane defines a plurality of pores having an average pore size of at least 40 kDa, 50 kDa or 60 kDa that permit inflammatory molecules in the blood to pass therethrough for removal from the blood. The blood depleted of inflammatory molecules is then returned to the recipient.

In another aspect, the invention provides a method of achieving a clinical endpoint indicative of a successful organ transplant in a recipient of a transplanted organ. In one embodiment, the method involves contacting blood from the recipient with an extracorporeal membrane. The extracorporeal membrane defines a plurality of pores having an average pore size of at least 40 kDa, 50 kDa or 60 kDa that permit inflammatory molecules in the blood to pass therethrough for removal from the blood. The blood depleted of inflammatory molecules is then returned to the recipient.

In yet another aspect, the invention provides a method of removing inflammatory molecules from the blood of a recipient of a transplanted organ by contacting blood from the recipient with an extracorporeal membrane defining a plurality of pores having an average pore size of at least 40 kDa, 50 kDa or 60 kDa to permit inflammatory molecules in the blood to pass through the pores for removal from the blood. The blood depleted of the inflammatory molecules is returned to the recipient.

In certain embodiments of any of the foregoing methods, the blood from the recipient is contacted with the extracorporeal membrane before, during, and/or after the transplant of the organ to the recipient. In certain embodiments, the pores are defined by a wall (e.g., an inner wall or an outer wall) of a semi-permeable hollow fiber.

In another aspect, the invention provides a method of improving a function of a transplanted organ in a recipient of the transplanted organ. In one embodiment, the method involves passing blood from the recipient through an extracorporeal cartridge comprising a housing and a plurality of semi-permeable hollow fibers, for example, substantially parallel semi-permeable hollow fibers, disposed therein. Each of the semi-permeable hollow fibers comprises a lumen and a plurality of pores in the walls of the hollow fibers such that when the blood traverses the lumens of the hollow fibers, inflammatory molecules from the blood pass through the pores and are removed from the blood. The blood depleted of inflammatory molecules is then returned to the recipient. The blood from the recipient is passed through the cartridge before, during, and/or after the transplant of the organ to the recipient.

In another aspect, the invention provides a method of treating or preventing primary graft dysfunction of a transplanted organ in a recipient of the transplanted organ. In one embodiment, the method involves passing blood from the recipient through an extracorporeal cartridge comprising a housing and a plurality of semi-permeable hollow fibers disposed therein. Each of the semi-permeable hollow fibers comprises a lumen and a plurality of pores in the walls of the hollow fibers such that when the blood traverses the lumens of the hollow fibers, inflammatory molecules from the blood pass through the pores and are removed from the blood. The blood depleted of inflammatory molecules is then returned to the recipient. The blood from the recipient is passed through the cartridge before, during, and/or after the transplant of the organ to the recipient.

In another aspect, the invention provides a method of treating or preventing acute rejection of a transplanted organ in a recipient of the transplanted organ. In one embodiment, the method involves passing blood from the recipient through an extracorporeal cartridge comprising a housing and a plurality of semi-permeable hollow fibers disposed therein. Each of the semi-permeable hollow fibers comprises a lumen and a plurality of pores in the walls of the hollow fibers such that when the blood traverses the lumens of the hollow fibers, inflammatory molecules from the blood pass through the pores and are removed from the blood. The blood depleted of inflammatory molecules is then returned to the recipient. The blood from the recipient is passed through the cartridge before, during, and/or after the transplant of the organ to the recipient.

In another aspect, the invention provides a method of treating or preventing delayed graft function of a transplanted organ in a recipient of the transplanted organ. In one embodiment, the method involves passing blood from the recipient through an extracorporeal cartridge comprising a housing and a plurality of semi-permeable hollow fibers disposed therein. Each of the semi-permeable hollow fibers comprises a lumen and a plurality of pores in the walls of the hollow fibers such that when the blood traverses the lumens of the hollow fibers, inflammatory molecules from the blood pass through the pores and are removed from the blood. The blood depleted of inflammatory molecules is then returned to the recipient. The blood from the recipient is passed through the cartridge before, during, and/or after the transplant of the organ to the recipient.

In another aspect, the invention provides a method of achieving a clinical endpoint indicative of a successful organ transplant in a recipient of a transplanted organ. In one embodiment, the method involves passing blood from the recipient through an extracorporeal cartridge comprising a housing and a plurality of semi-permeable hollow fibers disposed therein. Each of the semi-permeable hollow fibers comprises a lumen and a plurality of pores in the walls of the hollow fibers such that when the blood traverses the lumens of the hollow fibers, inflammatory molecules from the blood pass through the pores and are removed from the blood. The blood depleted of inflammatory molecules is then returned to the recipient. The blood from the recipient is passed through the cartridge before, during, and/or after the transplant of the organ to the recipient.

In yet another aspect, the invention provides a method of removing inflammatory molecules from the blood of a recipient of a transplanted organ by passing blood from the recipient through an extracorporeal cartridge comprising a housing and a plurality of semi-permeable hollow fibers defining a lumen and a plurality of pores, such that when the blood to traverses the lumens of the hollow fibers, inflammatory molecules from the blood pass through the pores and are removed from the blood. The blood depleted of the inflammatory molecules is returned to the recipient. The blood from the recipient is passed through the cartridge before, during, and/or after the transplant of the organ to the recipient.

In certain embodiments of any of the foregoing methods, the blood is contacted with the membrane or passed through the cartridge within 30 days prior to transplant of the organ. In another embodiment, the blood is contacted with the membrane or passed through the cartridge within 7 days prior to transplant of the organ. In yet another embodiment, the blood is contacted with the membrane or passed through the cartridge during a transplant surgery to transplant the organ. In a further embodiment, the blood is contacted with the membrane or passed through the cartridge after a transplant of the organ, for example, within 0.5-120 hours after a transplant of the organ.

In certain embodiments of any of the foregoing methods, the pores have an average pore size of from about 60 kDa to about 150 kDa. In other embodiments, the average pore size is greater than 65 kDa. In other embodiments, the average pore size is no greater than 65 kDa. In still further embodiments, the average pore size is from about 60 kDa to about 65 kDa. In some embodiments, the average pore size is about 40 kDa or greater. In some embodiments, the average pore size is about 50 kDa or greater. In other embodiments, the average pore size is no greater than 40 kDa. In other embodiments, the average pore size is no greater than 50 kDa. In other embodiments, the pores have an average pore size of from about 40 kDa to about 150 kDa. In other embodiments, the pores have an average pore size of from about 50 kDa to about 150 kDa.

In still further embodiments, the molecular weight cut-off the membrane or hollow fibers is about 65 kDa, where, for example, molecules greater than 65 kDa do not readily pass through the pores of the membrane or hollow fibers. In other embodiments, molecules less than 65 kDa can pass through the pores of the membrane or hollow fibers.

In certain embodiments of any of the foregoing methods, the recipient is a human, e.g., an adult human or a pediatric human. In certain embodiments, the organ or organs is one or more of a heart, lung, kidney, liver, intestine, or pancreas.

In certain embodiments of any of the foregoing methods, the membrane or the hollow fibers comprise a polymer. In certain embodiments, the polymer is polysulfone. In certain embodiments, the lumen of the hollow fibers has a diameter of from about 100 μM to about 700 μM. In certain embodiments, the diameter is from about 175 μM to about 225 μM. In another embodiment, the diameter is from about 600 μM to about 700 μM. In certain embodiments, the surface area of the hollow fibers is from about 0.01 m² to about 4.0 m², e.g., from about 1.9 m² to about 2.1 m², from about 0.05 m² to about 0.1 m², from about 0.25 m² to about 0.75 m², or from about 1.0 m² to 1.5 m².

In certain embodiments of any of the foregoing methods, the cartridge comprises a fluid inlet port and a fluid outlet port and/or the cartridge comprises one or more ultrafiltrate ports.

In certain embodiments of any of the foregoing methods, the inflammatory molecules removed from the blood are selected from one or more of IL-4, IL-6, IL-8, TNF-α, IL-1β, MCP-1, CCL2, IP-10, CXCL10, C3a, C5a, soluble TNF receptor II, soluble TNF receptor I, matrix metalloproteinase-9, matrix metalloproteinase-7, IL-10, soluble gp130, lipopolysaccharide (LPS), or procalcitonin. In certain embodiments, the inflammatory molecules removed from the blood are selected from one or more of IL-6, TNF-α, C3a, and C5a.

In certain embodiments of any of the foregoing methods, the flow rate of blood through the cartridge is from about 100 mL/min to about 600 mL/min. For example, in some embodiments, the flow rate is from about 100 mL/min to about 400 mL/min, from about 150 mL/min to about 300 mL/min, or from about 150 mL/min to about 250 mL/min.

In certain embodiments of any of the foregoing methods, the recipient's blood is passed through the cartridge or contacted with the membrane before the surgical procedure where the organ is transplanted into the subject. In certain embodiments of any of the foregoing methods, the recipient's blood is passed through the cartridge or contacted with the membrane during the surgical procedure where the organ is transplanted into the subject. In certain embodiments, the recipient's blood is passed through the cartridge or contacted with the membrane after the surgical procedure where the organ is transplanted into the subject (e.g., within about 12 hours, about 1 day, about 2 days, about 3 days, about 4 days, about 5 days, about 6 days, about 1 week, about 2 weeks, about 3 weeks, about 4 weeks, about 6 weeks, about 8 weeks, about 10 weeks, or about 12 weeks after the transplant of the organ to the recipient).

In certain embodiments of any of the foregoing methods, the recipient's blood is passed through the cartridge or contacted with the membrane for about 30 minutes to about 120 hours after the transplant of the organ to the recipient. For example, in certain embodiments, the recipient's blood is passed through the cartridge or contacted with the membrane for about 30 minutes to about 72 hours after the transplant of the organ to the recipient. For example, in some embodiments, the recipient's blood is passed through the cartridge or contacted with the membrane for from about 1 hour to about 72 hours, about 1 hour to about 24 hours, about 6 hours to about 12 hours, or about 3 hours to about 6 hours after the transplant of the organ to the recipient. In other embodiments, the recipient's blood is passed through the cartridge or contacted with the membrane for about 1 hour, about 3 hours, about 6 hours, about 9 hours, about 12 hours, about 24 hours, about 48 hours or about 72 hours after the transplant of the organ to the recipient.

In certain embodiments of any of the foregoing methods, the recipient's blood is passed through the cartridge or contacted with the membrane more than once, for example, 1, 2, 3, 4, 5, 6, 7, 8, 9, 10, 11, 12, 14, 16, 18, 20, 22, 24, or more than 24 times. In certain embodiments, the recipient's blood is passed through the cartridge or contacted with the membrane multiples times (e.g., repeatedly, e.g., at regular intervals) over a treatment period. For example, in certain embodiments, the recipient's blood is passed through the cartridge or contacted with the membrane for about 1 hour, about 2 hours, about 3 hours, about 4 hours, about 5 hours, about 6 hours, about 9 hours, or about 12 hours, about every 3 hours, about every 6 hours, about every 12 hours, about every day, about every 24 hours, about every 2 days, about every 3 days, about every 4 days, about every 5 days, about every 6 days or about every 7 days, over a period of about 1 day, about 2 days, about 3 days, about 4 days, about 5 days, about 6 days, about 1 week, about 2 weeks, about 3 weeks, about 4 weeks, about 6 weeks, about 8 weeks, about 10 weeks, or about 12 weeks.

In certain embodiments of any of the foregoing methods, the cartridge or membrane is connected to the recipient via an extracorporeal circuit or extracorporeal circulation system comprising a line from an artery of the recipient and a line to a vein of the recipient. In certain embodiments, the extracorporeal circuit or circulation system further comprises an ultrafiltrate collection line and/or container. In certain embodiments, the cartridge or membrane is connected to the recipient via an extracorporeal circuit or extracorporeal circulation system comprising a line from a vein of the recipient and a line to a vein of the recipient. In certain embodiments, the extracorporeal circuit or circulation system further comprises an ultrafiltrate collection line and/or container. In certain embodiments, the extracorporeal circuit or circulation system comprises a double lumen catheter inserted in to a vein of the recipient enabling pumping of blood from the vein and returning of blood to the vein. In certain embodiments, the extracorporeal circuit or circulation system further comprises one or more of an ultrafiltrate pump, ultrafiltrate pressure sensor, blood sensor, filter pressure sensor, venous pressure sensor, access pressure sensor, IV fluid return pump, ultrafiltration controller, or a temperature regulator.

In certain embodiments of any of the foregoing methods, the ultrafiltration rate of the cartridge is from about 1 mL/min to about 180 mL/min, e.g., from about 40 mL/min to about 180 mL/min.

In certain embodiments of any of the foregoing methods, (i) prior to and/or during the transplant, a first cartridge or membrane is connected to the recipient via an extracorporeal circuit comprising a line from an artery or vein of the recipient and a line to a vein of the recipient, and (ii) after the transplant, a second cartridge or membrane is connected to the recipient via an extracorporeal circuit comprising a line from an artery or vein of the recipient and a line to a vein of the recipient.

In certain embodiments, after the recipient's blood is passed through the cartridge or contacted with the membrane, the recipient shows an improvement in one or more clinical endpoints. For example, where the transplanted organ is a heart, in certain embodiments, the recipient may exhibit one or more of the following clinical endpoints: left ventricular ejection fraction (LVEF) stabilized at >40% (as measured by echocardiography); right arterial pressure (RAP) stabilized at <15 mm Hg; pulmonary capillary wedge pressure (PCWP) stabilized at <20 mm Hg; cardiac index (CI) stabilized at >2.0 L/min/m²; inotrope is not required or required only at low doses with an inotrope score <10; mean arterial pressure (MAP) stabilized at ≥70 mm Hg; no need for an intraaortic balloon pump (IABP), extracorporeal membrane oxygenation (ECMO), left ventricular assist device (LVAD), or biventricular assist or device (BiVAD); right arterial pressure (RAP) stabilized at <15 mm Hg; no need for right ventricular assist device (RVAD); lack of delayed graft function; lack of acute humoral or cellular rejection on endomyocardial biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and/or more rapid return to normal daily activities (relative to a recipient who has not been treated with a method of the invention).

For example, where the transplanted organ is a lung, in certain embodiments, the recipient may exhibit one or more of the following clinical endpoints: PaO₂/FiO₂ stabilized at >300; liberation from supplemental oxygen; lack of pathology on chest radiographs (or mild pathology which rapidly resolves); lack of delayed graft function; lack of humoral or cellular rejection on transbronchial, or other biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and/or more rapid return to normal daily activities (relative to a recipient who has not been treated with a method of the invention).

For example, where the transplanted organ is a kidney, in certain embodiments, the recipient may exhibit one or more of the following clinical endpoints: if placed on machine perfusion, low or declining resistive index to flow of perfusion fluid; increased or normalized glomerular filtration rate, as indicated by creatinine clearance rising or serum creatinine levels and blood urea nitrogen levels declining toward normal; lack of or resolved albuminuria; no requirement for renal replacement treatment in the first week after transplant; lack of acute humoral or cellular rejection on renal biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and/or more rapid return to normal daily activities (relative to a recipient who has not been treated with a method of the invention).

For example, where the transplanted organ is a liver, in certain embodiments, the recipient may exhibit one or more of the following clinical endpoints: on postoperative day 7 the concentration of bilirubin is less than 10 mg/dL; an international normalized ratio of less than 1.6; and alanine aminotransferase or aspartate aminotransferase levels not greater than 2000 IU/L within 7 postoperative days; if low serum sodium, an increase in serum sodium to ≥135 mEq/Liter; if high serum sodium, a decrease in serum sodium to ≤145 mEq/Liter; if elevated serum creatinine, a reduction or normalization of serum creatinine; lack of early allograph dysfunction; lack of acute humoral or cellular rejection on biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and/or more rapid return to normal daily activities (relative to a recipient who has not been treated with a method of the invention).

For example, where the transplanted organ is an intestine, in certain embodiments, the recipient may exhibit one or more of the following clinical endpoints: lack of early graft dysfunction; lack of acute humoral or cellular rejection on biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); less renal dysfunction (relative to a recipient who has not been treated with a method of the invention); and/or more rapid return to normal daily activities (relative to a recipient who has not been treated with a method of the invention).

For example, where the transplanted organ is a pancreas, in certain embodiments, the recipient may show one or more of the following endpoints: lack of early graft dysfunction, lack of acute humoral or cellular rejection on biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); less multiple organ failure (relative to a recipient who has not been treated with a method of the invention); less renal dysfunction (relative to a recipient who has not been treated with a method of the invention); and/or more rapid return to their normal daily activities (relative to a recipient who has not been treated with a method of the invention).

In certain embodiments of any of the foregoing methods, the methods of contacting blood from the recipient or passing blood from the transplant recipient through an extracorporeal cartridge are separate and distinct from any pre-operative, intra-operative, or post-operative renal replacement therapy (RRT) received by the recipient such as but not limited to continuous veno-venous hemofiltration (CVVH), continuations renal replacement therapy (CRRT) or dialysis, to prevent failure of the recipient's own kidneys incident to transplantation of an organ, such as, for example, a liver.

These and other aspects and features of the invention are described in the following detailed description and claims.

### DESCRIPTION OF THE DRAWINGS

The invention can be more completely understood with reference to the following drawings, in which

FIG. 1 is a schematic representation of an exemplary cartridge;

FIG. 2 is a schematic representation of an exemplary extracorporeal blood circuit used for a recipient of an organ transplant;

FIG. 3 is a schematic representation of an exemplary extracorporeal blood circuit used for a recipient of an organ transplant; and

FIG. 4 is a schematic representation of an exemplary extracorporeal blood circuit used for a recipient of an organ transplant.

### DETAILED DESCRIPTION

The invention is based, in part, upon the discovery that outcomes in a recipient of a transplanted organ can be improved by contacting blood from the recipient with a semi-permeable membrane, for example, one or more membranes disposed in a hemofilter, whereupon, inflammatory cytokines or other inflammatory molecules can pass through the semi-permeable membranes. Without wishing to be bound by theory, it is contemplated that processing the recipient blood using this approach removes inflammatory cytokines or other inflammatory molecules from the blood that negatively impact organ function.

Accordingly, in one aspect, the invention provides a method of improving a function of a transplanted organ in a recipient of the transplanted organ. In another aspect, the invention provides a method of treating or preventing primary graft dysfunction of a transplanted organ in a recipient of the transplanted organ. In another aspect, the invention provides a method of treating or preventing delayed graft function. In another aspect, the invention provides a method of treating or preventing acute rejection of a transplanted organ in a recipient of the transplanted organ. In another aspect, the invention provides a method of achieving a clinical endpoint indicative of a successful organ transplant in a recipient of a transplanted organ.

In certain embodiments, the methods of the invention involve exposing blood from the recipient to an extracorporeal membrane where the membrane comprises a plurality of pores that permit inflammatory molecules to be removed from the blood so that blood depleted of inflammatory molecules can be returned to the recipient.

In certain embodiments, the methods of the invention involve passing blood from the recipient through an extracorporeal cartridge comprising a housing and a plurality of semi-permeable hollow fibers disposed therein. Each of the semi-permeable hollow fibers defines a lumen and a plurality of pores, for example, in the wall of the hollow fiber, such that when the blood passes through the lumens of the hollow fibers, inflammatory molecules from the blood pass through the pores and are removed from the blood. Blood depleted of inflammatory molecules is returned to the recipient. In some embodiments, blood from the recipient is passed through the cartridge before, during and/or after the transplant of the organ to the recipient.

Various features and aspects of the invention are discussed in more detail below.

### I. Membranes/Cartridges

The methods of the invention relate to contacting blood from a recipient with an extracorporeal membrane. The membrane includes a plurality of pores having an average pore size of at least 40 kDa, 50 kDa, or 60 kDa that, for example, permit inflammatory molecules to be removed from the blood so that blood depleted of inflammatory molecules can be returned to the recipient.

In certain embodiments, the extracorporeal membrane is disposed in a cartridge. Although the underlying principles for designing an appropriate cartridge are discussed in detail, it is understood that cartridges useful in the practice of the invention are not limited to the particular design configurations discussed herein. In certain embodiments, a cartridge useful in the practice of the invention may, for example, comprise a housing and a plurality of semi-permeable hollow fibers disposed therein, each of the semi-permeable hollow fibers comprising a lumen and a plurality of pores. The cartridge may further comprise a fluid inlet port and a fluid outlet port and/or one or more ultrafiltrate ports.

Other cartridges useful in the practice of the invention include one or more fluid permeable membranes capable of filtering inflammatory molecules from the blood.

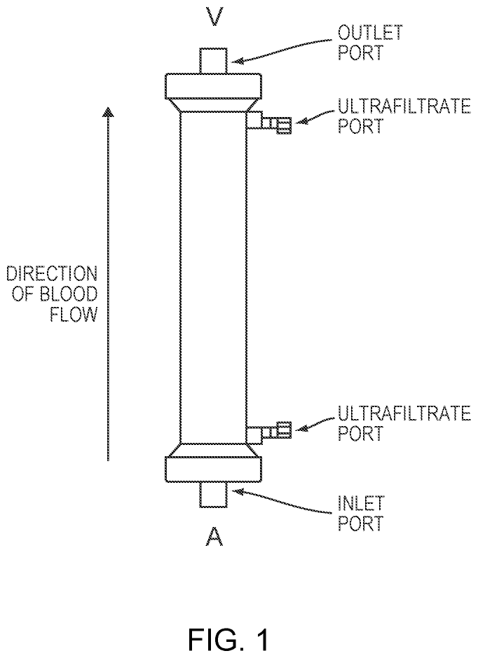

For example, as shown in FIG. 1, blood may enter the cartridge through a fluid inlet port (e.g., an arterial inlet port), pass through the hollow fibers and exit at the opposite end through a fluid outlet port (e.g., a venous outlet port).

It is understood that the membrane or hollow fibers in the cartridge used for filtration are not limited to a particular type, kind or size, and may be made of any appropriate material; however, the material should be biocompatible. For example, a surface of the membrane or fibers may be any biocompatible polymer comprising one or more of nylon, polyethylene, polyurethane, polyethylene terephthalate (PET), polytetrafluoroethylene (PTFE), CUPROPHAN (a cellulose regenerated by means of the cuprammonium process, available from Enka), HEMOPHAN (a modified CUPROPHAN with improved biocompatibility, available from Enka), CUPRAMMONIUM RAYON (a variety of CUPROPHAN, available from Asahi), BIOMEMBRANE (cuprammonium rayon available from Asahi), saponified cellulose acetate (such as fibers available from Teijin or CD Medical), cellulose acetate (such as fibers available from Toyobo Nipro), cellulose (such as that are regenerated by the modified cupramonium process or by means of the viscose process, available from Terumo or Textikombinat (Pirna, GDR) respectively), polyacrylonitrile (PAN), polysulfone, polyethersulfone, polyarylethersulfone, acrylic copolymers (such as acrylonitrile-NA-methallyl-sulfonate copolymer, available from Hospal), polycarbonate copolymer (such as GAMBRONE, a fiber available from Gambro), polymethylmethacrylate copolymers (such as fibers available from Toray), ethylene vinyl copolymer (such as EVAL, an ethylene-vinyl alcohol copolymer available from Kuraray), polyvinylalcohol, polyamide, and polycarbonate. Alternatively, a surface may be nylon mesh, cotton mesh, or woven fiber. The surface can have a constant thickness or an irregular thickness. In some embodiments, fibers may include silicon, for example, silicon nanofabricated membranes (see, e.g., U.S. Patent Publication No. 2004/0124147). In certain embodiments, the membrane or hollow fibers in the cartridge used for filtration include a polysulfone, e.g., glycerin-free polysulfone. Other suitable biocompatible fibers are known in the art, for example, in Salem and Mujais (1993) DIALYSIS THERAPY 2D ED., Ch. 5: Dialyzers, Eds. Nissensen and Fine, Hanley & Belfus, Inc., Philadelphia, Pa.

Depending upon the recipient and the application, the surface area of the membrane or hollow fibers in the cartridge used for filtration may be from about 0.01 m² to about 4.0 m². For example, the surface area of the hollow fibers may be from about 0.01 m² to about 3.0 m², about 0.01 m² to about 2.0 m², about 0.01 m² to about 1.0 m², about 0.01 m² to about 0.5 m², about 0.01 m² to about 0.1 m², about 0.01 m² to about 0.05 m², about 0.05 m² to about 4.0 m², about 0.05 m² to about 3.0 m², about 0.05 m² to about 2.0 m², about 0.05 m² to about 1.0 m², about 0.05 m² to about 0.5 m², about 0.05 m² to about 0.1 m², about 0.1 m² to about 4.0 m², about 0.1 m² to about 3.0 m², about 0.1 m² to about 2.0 m², about 0.1 m² to about 1.0 m², about 0.1 m² to about 0.5 m², about 0.2 m² to about 0.4 m², about 0.25 m² to about 0.35 m², about 0.5 m² to about 4.0 m², about 0.5 m² to about 3.0 m², about 0.5 m² to about 2.0 m², about 0.5 m² to about 1.0 m², about 0.5 m² to about 0.75 m², about 1.0 m² to about 4.0 m², about 1.0 m² to about 3.0 m², about 1.0 m² to about 2.0 m², about 1.0 m² to about 1.5 m², about 1.75 m² to about 2.5 m², about 1.75 m² to about 2.25 m², about 2.0 m² to about 4.0 m², about 2.0 m² to about 3.0 m², or about 3.0 m² to about 4.0 m². In certain embodiments, the surface area of the hollow fibers is from about 1.9 m² to about 2.1 m², from about 0.05 m² to about 0.1 m², from about 0.25 m² to about 0.75 m², or from about 1.0 m² to 1.5 m². In certain embodiments, the surface area is about 2.0 m². It will be appreciated that the surface area will vary depending on the age and size of the recipient. For example, pediatric and infant recipients will require cartridges with smaller surface areas as compared to adult recipients; smaller adults may also require cartridges with smaller surface areas as compared to larger adults.

The surface area of the membrane or hollow fibers can be adapted by lengthening or shortening the length of the membrane or fibers. For example, the length of the membrane or hollow fibers may be about 30 cm, about 29 cm, about 28 cm, about 27 cm, about 26 cm, about 25 cm, about 24 cm, about 23 cm, about 22 cm, about 21 cm, about 20 cm, about 19 cm, about 18 cm, about 17 cm, about 16 cm, about 15 cm, about 14 cm, about 13 cm, about 12 cm, about 11 cm, about 10 cm, or about 5 cm.

The surface area of the hollow fibers can also be adapted by varying the number of hollow fibers used in the cartridge. In certain embodiments, the cartridge comprises from about 9,000 to about 15,000 hollow fibers. For example, the cartridge may comprise from about 9,000 to about 14,000, from about 9,000 to about 13,000, from about 9,000 to about 12,000, from about 9,000 to about 11,000, from about 9,000 to about 10,000, from about 10,000 to about 15,000, from about 10,000 to about 14,000, from about 10,000 to about 13,000, from about 10,000 to about 12,000, from about 10,000 to about 11,000, from about 11,000 to about 15,000, from about 11,000 to about 14,000, from about 11,000 to about 13,000, from about 11,000 to about 12,000, from about 12,000 to about 15,000, from about 12,000 to about 14,000, from about 12,000 to about 13,000, from about 12,000 to about 15,000, from about 12,000 to about 14,000, or from about 14,000 to about 13,000 hollow fibers.

Also depending upon the recipient and the application, the lumen of hollow fibers in the cartridge may be from about 100 μM to about 700 μM. For example, the lumen may be from about 100 μM to about 700 μM, about 100 μM to about 600 μM, about 100 μM to about 500 μM, about 100 μM to about 400 μM, about 100 μM to about 300 μM, about 100 μM to about 200 μM, about 200 μM to about 700 μM, about 200 μM to about 600 μM, about 200 μM to about 500 μM, about 200 μM to about 400 μM, about 200 μM to about 300 μM, about 300 μM to about 700 μM, about 300 μM to about 600 μM, about 300 μM to about 500 μM, about 300 μM to about 400 μM, about 400 μM to about 700 μM, about 400 μM to about 600 μM, about 400 μM to about 500 μM, about 500 μM to about 700 μM, about 500 μM to about 600 μM, or about 600 μM to about 700 μM. In certain embodiments, the lumen of the hollow fibers has a diameter of about 175 μM to about 225 μM, or about 600 μM to about 700 μM. In certain embodiments, the lumen of the hollow fibers has a diameter of about 200 μM.

In certain embodiments, the hollow fibers are made of a semi-permeable membrane. The term “membrane” refers to a surface capable of receiving a fluid on both sides of the surface, or a fluid on one side and gas on the other side of the surface. It is understood that the sieving characteristics of a membrane depend not only on the pore size, but also on the physical, chemical, and electrical characteristics of the material from which the fiber or membrane is made, the particular manufacturing technique used and post production processing (e.g. sterilization). Nonetheless, the size of a pore in a porous membrane or fiber can be represented by a molecular weight cutoff (MWC or MWCO), i.e., the lowest molecular weight of solute in which 90% of the solute is retained by the membrane or fiber. Molecular weight cutoff may be measured by any method known in the art, including, for example, exposing the membrane or fiber to a solute with a known molecular weight (e.g., a polyethylene glycol or dextran) and ascertaining retention of the solute by the membrane or fiber. It is understood that the molecular weight cutoff may vary depending upon the conditions in which it is measured, for example, the molecular weight cutoff of a membrane or fiber that is measured when the membrane or fiber is disposed in an extracorporeal circuit including subject blood (i.e. the effective molecular weight cutoff) may be lower than the molecular weight cutoff of the membrane or fiber that is measured in a test situation (i.e. the nominal effective molecular weight cutoff). Further, flow rates, temperature, fluid type, and duration of use can impact the measurement of the molecular weight cutoff of a given membrane or fiber.

In certain embodiments, a molecular weight cutoff or pore size of a membrane or fiber refers to the molecular weight cutoff or pore size of the membrane or fiber that is measured when the membrane or fiber is disposed in a circuit (e.g., an extracorporeal circuit) including subject blood. In certain embodiments, a molecular weight cutoff or pore size of a membrane or fiber refers to the molecular weight cutoff or pore size of the membrane or fiber that is measured when the membrane or fiber is disposed in a circuit (e.g., an extracorporeal circuit) including subject plasma. In certain embodiments, a molecular weight cutoff or pore size of a membrane or fiber refers to the molecular weight cutoff or pore size of the membrane or fiber that is measured when the membrane or fiber is disposed in a circuit including water (e.g., aqueous saline). In certain embodiments, a molecular weight cutoff or pore size of a membrane or fiber refers to the molecular weight cutoff or pore size of the membrane or fiber that is measured when the membrane or fiber is disposed in a circuit (e.g., an extracorporeal circuit) including subject blood or plasma, and does not refer to the molecular weight cutoff or pore size of the membrane or fiber that is measured when the membrane or fiber is disposed in a circuit including water (e.g., aqueous saline).

A membrane or fiber can be porous (e.g., selectively porous or semi-porous) such that it is capable of fluid or gas flow therethrough. It is understood that the term “porous” as used herein to describe a surface, fiber, or membrane includes generally porous, selectively porous and/or semi-porous surfaces or membranes. A semi-permeable membrane refers to a membrane that permits only certain molecules to pass through while being impermeable to other molecules. In one embodiment, a membrane is semi-permeable based on the size of molecules contacting the membrane. For example, in one embodiment, a semi-permeable membrane is permeable to molecules below a certain size threshold while molecules above that size threshold are excluded from passing through the membrane.

Accordingly, in certain embodiments, the semi-permeable membrane or hollow fibers in the cartridge used for filtration of recipient blood comprise a plurality of pores with an average pore size of from about 60 kDa to about 150 kDa. For example, the plurality of pores may have an average pore size of about 65 kDa to about 150 kDa, about 70 kDa to about 150 kDa, about 80 kDa to about 150 kDa, about 90 kDa to about 150 kDa, about 100 kDa to about 150 kDa, about 110 kDa to about 150 kDa, about 120 kDa to about 150 kDa, about 130 kDa to about 150 kDa, about 140 kDa to about 150 kDa, about 60 kDa to about 140 kDa, about 65 kDa to about 140 kDa, about 70 kDa to about 140 kDa, about 80 kDa to about 140 kDa, about 90 kDa to about 140 kDa, about 100 kDa to about 140 kDa, about 110 kDa to about 140 kDa, about 120 kDa to about 140 kDa, about 130 kDa to about 140 kDa, about 60 kDa to about 130 kDa, about 65 kDa to about 130 kDa, about 70 kDa to about 130 kDa, about 80 kDa to about 130 kDa, about 90 kDa to about 130 kDa, about 100 kDa to about 130 kDa, about 110 kDa to about 130 kDa, about 120 kDa to about 130 kDa, about 60 kDa to about 120 kDa, about 65 kDa to about 120 kDa, about 70 kDa to about 120 kDa, about 80 kDa to about 120 kDa, about 90 kDa to about 120 kDa, about 100 kDa to about 120 kDa, about 110 kDa to about 120 kDa, about 60 kDa to about 110 kDa, about 65 kDa to about 110 kDa, about 70 kDa to about 110 kDa, about 80 kDa to about 110 kDa, about 90 kDa to about 110 kDa, about 100 kDa to about 110 kDa, about 60 kDa to about 100 kDa, about 65 kDa to about 100 kDa, about 70 kDa to about 100 kDa, about 80 kDa to about 100 kDa, about 90 kDa to about 100 kDa, about 60 kDa to about 90 kDa, about 65 kDa to about 90 kDa, about 70 kDa to about 90 kDa, about 80 kDa to about 90 kDa, about 60 kDa to about 80 kDa, about 65 kDa to about 80 kDa, about 70 kDa to about 80 kDa, about 60 kDa to about 70 kDa, about 65 kDa to about 70 kDa, or about 60 kDa to about 65 kDa. The plurality of pores may have an average pore size greater than 60 kDa. The plurality of pores may have an average pore size greater than 65 kDa. The plurality of pores may have an average pore size greater than 70 kDa. The plurality of pores may have an average pore size greater than 80 kDa, greater than 90 kDa, greater than 100 kDa, greater than 110 kDa, greater than 120 kDa, greater than 130 kDa, greater than 140 kDa, or greater than 150 kDa. The plurality of pores may have an average pore size no greater than 65 kDa. The plurality of pores may have an average pore size from about 60 kDa to about 65 kDa. The plurality of pores may have an average pore size from about 40 kDa to 150 kDa. The plurality of pores may have an average pore size from about 50 kDa to 150 kDa. The plurality of pores may have an average pore size of about 40 kDa or greater. The plurality of pores may have an average pore size of about 50 kDa or greater. The plurality of pores may have an average pore size of not greater than 40 kDa. The plurality of pores may have an average pore size of not greater than 50 kDa.

In certain embodiments, the molecular weight cut-off the membrane or hollow fibers is about 65 kDa, where, for example, molecules greater than 65 kDa do not readily pass through the pores of the membrane or hollow fibers. In other embodiments, molecules less than 65 kDa can pass through the pores of the membrane or hollow fibers.

As used herein, the sieving coefficient (SC) of a membrane or fiber for a given solute refers to the ratio between the solute concentration in the filtrate and its concentration in the feed (e.g., blood, plasma, plasma water, or water). An SC of 1 indicates unrestricted transport while an SC of 0 indicates no transport at all. SC is specific for each fiber or membrane for each solute. It is understood that SC varies depending upon the treatment conditions, and measurement of the SC may even vary during treatment because the characteristics of the fiber or membrane may change.

In certain embodiments, the membrane or hollow fibers in the cartridge used for filtration have a sieving coefficient for IL-6 of about 0.8 to about 1.0. For example, the sieving coefficient for IL-6 may be from about 0.1 to about 1.0, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 1.0, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 1.0, about 0.3 to about 0.9, about 0.3 to about 0.8, about 0.3 to about 0.7, about 0.3 to about 0.6, about 0.3 to about 0.5, about 0.3 to about 0.4, about 0.4 to about 1.0, about 0.4 to about 0.9, about 0.4 to about 0.8, about 0.4 to about 0.7, about 0.4 to about 0.6, about 0.4 to about 0.5, about 0.5 to about 1.0, about 0.5 to about 0.9, about 0.5 to about 0.8, about 0.5 to about 0.7, about 0.5 to about 0.6, about 0.6 to about 1.0, about 0.6 to about 0.9, about 0.6 to about 0.8, about 0.6 to about 0.7, about 0.7 to about 1.0, about 0.7 to about 0.9, about 0.7 to about 0.8, about 0.8 to about 1.4, about 0.8 to about 1.0, about 0.8 to about 0.9, or about 0.9 to about 1.0. In certain embodiments, the sieving coefficient for IL-6 is at least 0.1, 0.2, 0.3, 0.4, 0.5, 0.6, 0.7, 0.8, 0.9, or 1.0 In certain embodiments, the sieving coefficient for IL-6 is about 1.0. In certain embodiments, the sieving coefficient for IL-6 is about 0.9. In certain embodiments, the sieving coefficient for IL-6 is greater than or equal to 0.1. In certain embodiments, the sieving coefficient for IL-6 may be measured as described in Clar et al. (1997) ASAIO J., 43:163-170.

In certain embodiments, the membrane or hollow fibers in the cartridge used for filtration have a sieving coefficient for urea of about 0.8 to about 1.0. For example, the sieving coefficient for urea may be from about 0.8 to about 1.0, from about 0.8 to about 0.9, or from about 0.9 to about 1.0. In certain embodiments, the sieving coefficient for urea is at least 0.8, 0.9, or 1.0. In certain embodiments, the sieving coefficient for urea is about 1.0. In certain embodiments, the sieving coefficient for urea may be measured in aqueous solution at a flow rate of 200 mL/min and transmembrane pressure (TMP) of 50 mmHg.

In certain embodiments, the membrane or hollow fibers in the cartridge used for filtration have a sieving coefficient for creatinine of about 0.8 to about 1.0. For example, the sieving coefficient for creatinine may be from about 0.8 to about 1.0, from about 0.8 to about 0.9, or from about 0.9 to about 1.0. In certain embodiments, the sieving coefficient for creatinine is at least 0.8, 0.9, or 1.0. In certain embodiments, the sieving coefficient for creatinine is about 1.0. In certain embodiments, the sieving coefficient for creatinine may be measured in aqueous solution at a flow rate of 200 mL/min and transmembrane pressure (TMP) of 50 mmHg.

In certain embodiments, the membrane or hollow fibers in the cartridge used for filtration have a sieving coefficient for vitamin B12 of about 0.8 to about 1.0. For example, the sieving coefficient for vitamin B12 may be from about 0.8 to about 1.0, from about 0.8 to about 0.9, or from about 0.9 to about 1.0. In certain embodiments, the sieving coefficient for creatinine vitamin B12 is at least 0.8, 0.9, or 1.0. In certain embodiments, the sieving coefficient for vitamin B12 is about 1.0. In certain embodiments, the sieving coefficient for vitamin B12 may be measured in aqueous solution at a flow rate of 200 mL/min and transmembrane pressure (TMP) of 50 mmHg.

In certain embodiments, the membrane or hollow fibers in the cartridge used for filtration have a sieving coefficient for myoglobin of about 0.10 to about 1.0. For example, the sieving coefficient for myoglobin may be from about 0.1 to about 1.0, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 1.0, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 1.0, about 0.3 to about 0.9, about 0.3 to about 0.8, about 0.3 to about 0.7, about 0.3 to about 0.6, about 0.3 to about 0.5, about 0.3 to about 0.4, about 0.4 to about 1.0, about 0.4 to about 0.9, about 0.4 to about 0.8, about 0.4 to about 0.7, about 0.4 to about 0.6, about 0.4 to about 0.5, about 0.5 to about 1.0, about 0.5 to about 0.9, about 0.5 to about 0.8, about 0.5 to about 0.7, about 0.5 to about 0.6, about 0.6 to about 1.0, about 0.6 to about 0.9, about 0.6 to about 0.8, about 0.6 to about 0.7, about 0.7 to about 1.0, about 0.7 to about 0.9, about 0.7 to about 0.8, about 0.8 to about 1.4, about 0.8 to about 1.0, about 0.8 to about 0.9, or about 0.9 to about 1.0. In certain embodiments, the sieving coefficient for myoglobin is at least 0.1, 0.2, 0.3, 0.4, 0.5, 0.6, 0.7, 0.8, 0.9, or 1.0. In certain embodiments, the sieving coefficient for myoglobin is about 1.0. In certain embodiments, the sieving coefficient for myoglobin is about 0.9. In certain embodiments, the sieving coefficient for myoglobin is greater than or equal to 0.1. In certain embodiments, the sieving coefficient for myoglobin is greater than or equal to 0.3 or is about 0.3 to about 0.4. In certain embodiments, the sieving coefficient for myoglobin is at least 0.10, 0.15, 0.20, or 0.25. In certain embodiments, the sieving coefficient for myoglobin is about 0.17. In certain embodiments, the sieving coefficient for myoglobin is about 0.1 to about 0.25. In certain embodiments, the sieving coefficient for myoglobin may be measured in bovine blood at a flow rate of 400 mL/min and transmembrane pressure (TMP) of 400 mmHg.

In certain embodiments, the membrane or hollow fibers in the cartridge used for filtration have a sieving coefficient for albumin of about 0.005 to about 0.025. For example, the sieving coefficient for albumin may be from about 0.005 to about 0.025, about 0.005 to about 0.020, about 0.005 to about 0.015, about 0.005 to about 0.010, about 0.010 to about 0.025, about 0.010 to about 0.020, about 0.010 to about 0.015, about 0.015 to about 0.025, about 0.015 to about 0.020, or about 0.020 to about 0.025. In certain embodiments, the sieving coefficient for albumin is at least 0.005, 0.010, 0.015, 0.020, or 0.025. In certain embodiments, the sieving coefficient for albumin is about 0.015. In certain embodiments, the sieving coefficient for albumin is less than 0.1. In certain embodiments, the membrane or hollow fibers in the cartridge used for filtration have a sieving coefficient for albumin of about 0.2 to about 1.0. For example, the sieving coefficient for albumin may be from about 0.2 to about 1.0, about 0.2 to about 0.8, about 0.2 to about 0.6, about 0.2 to about 0.4, about 0.4 to about 1.0, about 0.4 to about 0.8, about 0.4 to about 0.6, about 0.6 to about 1.0, about 0.6 to about 0.8, or about 0.8 to about 1.0. In certain embodiments, the sieving coefficient for albumin is about 0.1 to about 1.0, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 1.0, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 1.0, about 0.3 to about 0.9, about 0.3 to about 0.8, about 0.3 to about 0.7, about 0.3 to about 0.6, about 0.3 to about 0.5, about 0.3 to about 0.4, about 0.4 to about 1.0, about 0.4 to about 0.9, about 0.4 to about 0.8, about 0.4 to about 0.7, about 0.4 to about 0.6, about 0.4 to about 0.5, about 0.5 to about 1.0, about 0.5 to about 0.9, about 0.5 to about 0.8, about 0.5 to about 0.7, about 0.5 to about 0.6, about 0.6 to about 1.0, about 0.6 to about 0.9, about 0.6 to about 0.8, about 0.6 to about 0.7, about 0.7 to about 1.0, about 0.7 to about 0.9, about 0.7 to about 0.8, about 0.8 to about 1.4, about 0.8 to about 1.0, about 0.8 to about 0.9, or about 0.9 to about 1.0. In certain embodiments, the sieving coefficient for albumin may be measured in bovine blood at a flow rate of 400 mL/min and transmembrane pressure (TMP) of 400 mmHg.

In methods where the sieving coefficient of albumin exceeds 0.1, the organ recipient may receive albumin exchange intravenously to compensate for albumin loss. For example, the albumin may be recombinant albumin or ultrapure albumin. The recombinant albumin may be human or of another mammal. The ultrapure albumin may be mammalian ultrapure albumin, e.g., from a pig, horse, or cow. Recombinant human albumin suitable for exchange therapy is available from InVitria (Aurora, Colo.), AkronBiotech (Boca Raton, Fla.), and Valley Biomedical Products & Services, Inc. (Winchester, Va.). It is believed that recombinant or ultrapure albumin exchange may enhance the methods of the invention as the antioxidant potential of such albumin is enhanced compared to albumin sourced from human or mammalian plasma and therefore is able to bind more inflammatory mediators, cytokines, and toxins, removing them from the subject's blood.

The housing of the cartridge is not limited to a particular set of dimensions (e.g., length, width, weight, or another dimension). It is understood that the size and shape of the housing of the cartridge may be designed to provide the appropriate fill volume and to minimize turbulence when a fluid is passed through the cartridge. Furthermore, it is understood that the size, shape and composition of the membrane located within the cartridge may be designed to provide the appropriate surface area and to minimize turbulence when a fluid is passed through the cartridge. Also, the size of the cartridge depends upon the size of the recipient. For example, pediatric and infant recipients will require smaller cartridges areas as compared to adult recipients; smaller adults may also require smaller cartridges as compared to larger adults.

The housing of the cartridge can be fabricated from a variety of materials, but the material that defines that fluid contacting surface in the inner volume should be biocompatible. The cartridge can be constructed from a variety of materials including, metals such as titanium, or stainless steel with or without surface coatings of refractory metals including titanium, tantalum, or niobium; ceramics such as alumina, silica, or zirconia; polymers, such as polyvinylchloride, polyethylene, or polycarbonate; or plastic.

It is understood that the cartridge, once fabricated, should be sterilized prior to use. Sterility can be achieved through exposure to one or more sterilizing agents, separately or in combination, such as high temperature, high pressure, radiation, or chemical agents such as ethylene oxide, for example. The cartridge preferably is sterilized once it has been packaged, for example, after it has been hermetically sealed within an appropriate container (i.e., the cartridge is terminally sterilized). The sterilization process preferably achieves a sterility assurance level (SAL) of 10⁻³ or less; i.e., the probability of any given unit being nonsterile after the process is no more than 1 in 10³. More preferably, the sterilization process achieves an SAL of no more than 10⁻⁴, no more than 10⁻⁵, or no more than 10⁻⁶.

In certain embodiments, an extracorporeal cartridge useful in the methods described herein is the CLR 2.0 filter (SeaStar Medical, Inc., Denver, Colo.). In certain embodiments, cartridge (e.g., a CLR 2.0 cartridge) comprises semipermeable polysulfone hollow fibers that have a molecular weight cut-off of about 65 kDa.

### II. Blood Circuits

It is understood that a membrane or cartridge can be used in a variety of different fluid circuits or extracorporeal circulation system depending upon the intended use. The fluid circuits generally are configured to improve organ transplant outcomes in a recipient of the transplanted organ.

In basic form, an exemplary circuit includes a cartridge, a fluid connection for blood to flow from a blood source (for example, an artery or vein in a subject, such as an organ recipient) to the cartridge, and a fluid connection for treated blood to flow from the cartridge to a receptacle (for example, back to a vein in the organ recipient).

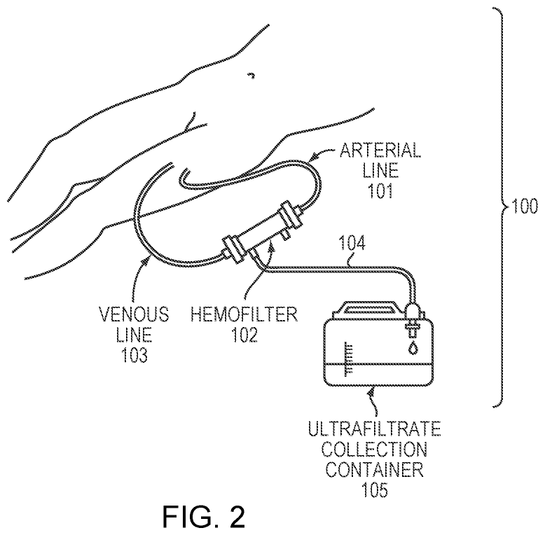

For example, as shown in the exemplary circuit 100 depicted in FIG. 2, blood flows from an artery in a recipient to a blood line 101. Blood then enters the cartridge 102, passes through the hollow fibers (which run parallel to the length of the cartridge), and exits at the opposite end. Blood then flows through a blood line 103 and is returned to a vein in the recipient. Hemofiltration occurs while the blood passes through the cartridge 102, with ultrafiltrate leaving the cartridge and passing through a line 104 to, for example, an ultrafiltrate collection container 105. During hemofiltration, plasma water and non-protein bound plasma solutes, including inflammatory molecules, are removed from the blood by ultrafiltration, while cellular elements and larger proteins are returned to the recipient. For an exemplary cartridge, ultrafiltration occurs as a result of pressure gradients across the porous hollow fiber membrane. This gradient is achieved by: (1) positive pressure in the blood compartment, provided by the recipient's mean arterial blood pressure and (2) negative pressure in the filtrate compartment, provided by the modest siphoning effect generated when ultrafiltrate is collected from the hemofilter (for example, in a container placed below the hemofilter). Replacement or substitution fluid may additionally be infused into the circuit 100 at a controlled rate to maintain fluid, electrolyte, acid base and nitrogen balance.

In certain embodiments, the extracorporeal circulation system further comprises one or more of an ultrafiltrate pump, ultrafiltrate pressure sensor, blood sensor, filter pressure sensor, venous pressure sensor, access pressure sensor, IV fluid return pump, ultrafiltration controller, or a temperature regulator.

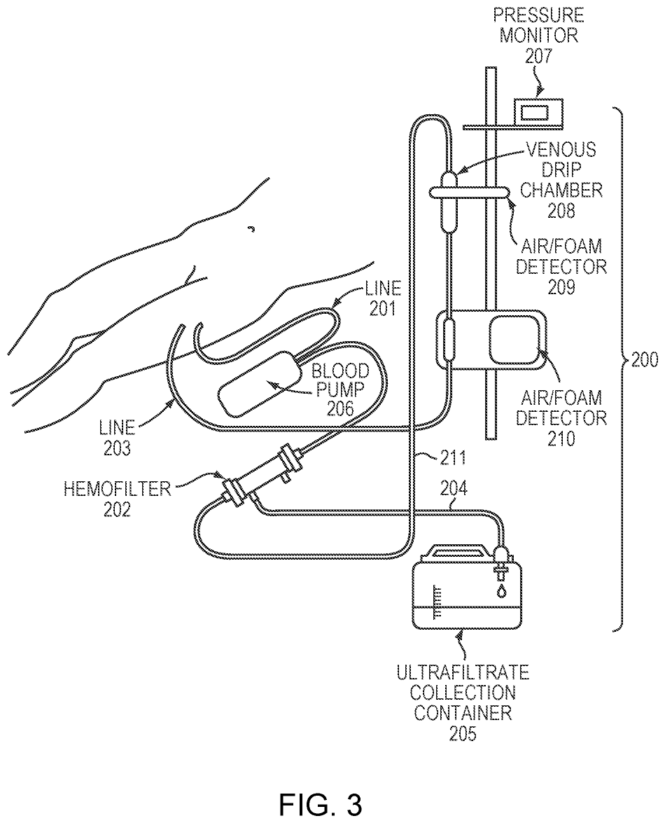

For example, in certain embodiments, a minimum flow rate is required for proper operation of the cartridge, and therefore one or more pumps may be necessary in recipients with systolic blood pressures below a certain threshold. For example, a pump assisted circuit 200 is shown in FIG. 3. Blood flows from an artery or vein in a recipient to a blood line 201. Blood enters a pump 206 before continuing to the cartridge 202. Flow rates at the pump 206 can be chosen at ranges described herein. Blood enters the cartridge 202, passes through the hollow fibers, and exits at the opposite end. Hemofiltration occurs while the blood passes through the cartridge, with ultrafiltrate leaving the cartridge and passing through a line 204 to, for example, an ultrafiltrate collection container 205. Blood then flows through a blood line 211 and, prior to returning to a vein in the recipient, passes through a venous drip chamber 208. The system may also include a pressure monitor 209 and air/foam detector 220.

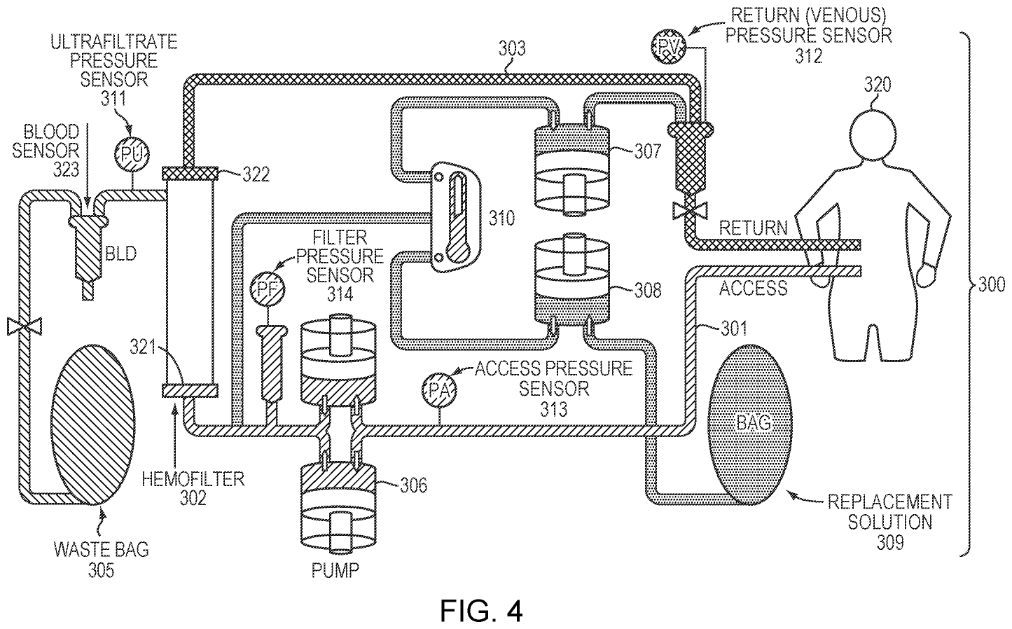

An additional exemplary circuit 300 is shown in FIG. 4. Blood flows from a recipient 300 to a blood line 301. Blood enters a pump 306 before continuing to the cartridge 302. Flow rates at the pump 306 can be chosen at ranges described herein. Pressure is sampled at an access pressure sensor 313 prior to the pump, and a filter pressure sensor 314 after the pump and prior to the cartridge 302. Blood enters the cartridge 302 via a fluid inlet port 321, passes through the hollow fibers, and exits at the opposite end via a fluid outlet port 322. Hemofiltration occurs while the blood passes through the cartridge 302, with ultrafiltrate leaving the cartridge and passing through an ultrafiltrate line 304 to, for example, a waste bag 305. The ultrafiltrate line 304 may include an ultrafiltrate pressure sensor 311 and/or a blood sensor 323. Filtered blood leaves the cartridge 302 and flows through a blood line 303 to the recipient. Pressure may be sampled at a return pressure sensor 312 prior to the returning to the recipient. The system may also include a source of replacement solution. Replacement solution flows from a bag 309 to a pump 308, and is introduced into the circuit immediately prior to the cartridge and/or immediately prior to the return of fluid to the recipient. Replacement solution temperature is monitored by a temperature regulator 310.

A cartridge may be connected to the recipient's vascular system via vascular access which may include: arteriovenous femoral catheters, arteriovenous jugular catheters, Quinton-Scribner Shunt, arteriovenous fistula, veno-venous femoral catheters, veno-venous jugular catheters, veno-venous subclavian catheters, or veno-venous catheters at other sites. In certain embodiments, this is accomplished with a percutaneous catheter arrangement or an arteriovenous shunt. In certain embodiments, the extracorporeal circulation system comprises a double lumen catheter inserted in to a vein enabling pumping of blood from the vein and returning of blood to the vein.

The rate of blood flowing through the system will depend on the condition of the recipient, the molecular weight cutoff of the associated fibers, the body size of recipient, and other requirements for effective preparation of organs for transplant. The amount of blood, the blood flow rate and the duration of treatment are preferably determined on a case by case basis after factoring the weight, the age and the nature of the recipient.

In certain embodiments, the blood flow rate through the cartridge is from about 100 mL/min to about 600 mL/min. For example the blood flow rate may be from about 200 mL/min to about 600 mL/min, about 300 mL/min to about 600 mL/min, about 400 mL/min to about 600 mL/min, about 500 mL/min to about 600 mL/min, about 100 mL/min to about 500 mL/min, about 200 mL/min to about 500 mL/min, about 300 mL/min to about 500 mL/min, about 400 mL/min to about 500 mL/min, about 100 mL/min to about 400 mL/min, about 200 mL/min to about 400 mL/min, about 300 mL/min to about 400 mL/min, about 100 mL/min to about 300 mL/min, about 200 mL/min to about 300 mL/min, or about 100 mL/min to about 200 mL/min. In certain embodiments, the blood flow rate is from about 100 mL/min to about 400 mL/min. In certain embodiments, the blood flow rate is from about 150 mL/min to about 250 mL/min. In certain embodiments, the blood flow rate is from about 135 mL/min to about 150 mL/min.

In certain embodiments, the ultrafiltration rate of the cartridge is from about 0 mL/min to about 180 mL/min, e.g., about 1 mL/min to about 180 mL/min. For example the ultrafiltration rate may be from about 1 mL/min to about 180 mL/min, about 5 mL/min to about 180 mL/min, about 20 mL/min to about 180 mL/min, about 40 mL/min to about 180 mL/min, about 60 mL/min to about 180 mL/min, about 80 mL/min to about 180 mL/min, about 100 mL/min to about 180 mL/min, about 120 mL/min to about 180 mL/min, about 140 mL/min to about 180 mL/min, about 160 mL/min to about 180 mL/min, about 1 mL/min to about 160 mL/min, about 5 mL/min to about 160 mL/min, about 20 mL/min to about 160 mL/min, about 40 mL/min to about 160 mL/min, about 60 mL/min to about 160 mL/min, about 80 mL/min to about 160 mL/min, about 100 mL/min to about 160 mL/min, about 120 mL/min to about 160 mL/min, about 140 mL/min to about 160 mL/min, about 1 mL/min to about 140 mL/min, about 5 mL/min to about 140 mL/min, about 20 mL/min to about 140 mL/min, about 40 mL/min to about 140 mL/min, about 60 mL/min to about 140 mL/min, about 80 mL/min to about 140 mL/min, about 100 mL/min to about 140 mL/min, about 120 mL/min to about 140 mL/min, about 1 mL/min to about 120 mL/min, about 5 mL/min to about 120 mL/min, about 20 mL/min to about 120 mL/min, about 40 mL/min to about 120 mL/min, about 60 mL/min to about 120 mL/min, about 80 mL/min to about 120 mL/min, about 100 mL/min to about 120 mL/min, about 1 mL/min to about 100 mL/min, about 5 mL/min to about 100 mL/min, about 20 mL/min to about 100 mL/min, about 40 mL/min to about 100 mL/min, about 60 mL/min to about 100 mL/min, about 80 mL/min to about 100 mL/min, about 1 mL/min to about 80 mL/min, about 5 mL/min to about 80 mL/min, about 20 mL/min to about 80 mL/min, about 40 mL/min to about 80 mL/min, about 60 mL/min to about 80 mL/min, about 1 mL/min to about 60 mL/min, about 5 mL/min to about 60 mL/min, about 20 mL/min to about 60 mL/min, about 40 mL/min to about 60 mL/min, about 1 mL/min to about 40 mL/min, about 5 mL/min to about 40 mL/min, about 20 mL/min to about 40 mL/min, about 1 mL/min to about 20 mL/min, about 5 mL/min to about 20 mL/min, or about 1 mL/min to about 5 mL/min. In certain embodiments, the ultrafiltration rate of the cartridge is from about 40 mL/min to about 180 mL/min.

In certain embodiments, the recipient's blood is passed through the cartridge or contacted with the membrane before the transplant of the organ to the recipient. In one embodiment, the recipient's blood is passed through the cartridge or contacted with the membrane before the transplant of a heart and/or lungs. In one embodiment, the recipient's blood is passed through the cartridge or contacted with the membrane before the transplant of an organ that is not a liver. In one embodiment, the recipient's blood is passed through the cartridge or contacted with the membrane before the transplant of an organ that is not a kidney. In one embodiment, the recipient's blood is passed through the cartridge or contacted with the membrane before the transplant of a kidney or liver. In certain embodiments, the recipient's blood is passed through the cartridge or contacted with the membrane during the transplant of the organ to the recipient. In certain embodiments, the recipient's blood is passed through the cartridge or contacted with the membrane after the transplant of the organ to the recipient. In certain embodiments, the recipient's blood is passed through the cartridge or contacted with the membrane during and after the transplant of the organ to the recipient. As used herein, “before the transplant of the organ to the recipient,” refers to any time before the surgical procedure where the organ is transplanted into the subject. As used herein, “during the transplant of the organ to the recipient,” refers to any portion or all of the duration of the surgical procedure where the organ is transplanted into the subject. As used herein, “after the transplant of the organ to the recipient,” refers to any time after the completion of surgical procedure where the organ is transplanted into the subject.

In certain embodiments, during the transplant of the organ to the recipient, the recipient's blood is passed through the cartridge or contacted with the membrane for from about 0.5 to about 24 hours. For example, the recipient's blood may be passed through the cartridge from about 0.5 to about 24, from about 0.5 to about 12, from about 0.5 to about 6, from about 0.5 to about 3, from about 0.5 to about 1, from about 1 to about 24, from about 1 to about 12, from about 1 to about 6, from about 1 to about 3, from about 3 to about 24, from about 3 to about 12, from about 3 to about 6, from about 6 to about 24, from about 6 to about 12, or from about 12 to about 24 hours. In certain embodiments, during the transplant of the organ to the recipient, the recipient's blood is passed through the cartridge or contacted with the membrane for about 1 hour, about 1 to about 3 hours, about 3 to about 6 hours, or about 6 to about 12 hours. In certain embodiments, during the transplant of the organ to the recipient, the recipient's blood is passed through the cartridge or contacted with the membrane for about 1 hour, about 3 hours, about 6 hours, about 9 hours, or about 12 hours.

In certain embodiments, after the transplant of the organ to the recipient, the recipient's blood is passed through the cartridge or contacted with the membrane for from about 0.5 to about 120 hours. For example, the recipient's blood may be passed through the cartridge from about 0.5 to about 120, from about 0.5 to about 96, from about 0.5 to about 72, from about 0.5 to about 48, from about 0.5 to about 24, from about 0.5 to about 12, from about 0.5 to about 6, from about 0.5 to about 3, from about 0.5 to about 1, from about 1 to about 120, from about 1 to about 96, from about 1 to about 72, from about 1 to about 48, from about 1 to about 24, from about 1 to about 12, from about 1 to about 6, from about 1 to about 3, from about 3 to about 120, from about 3 to about 96, from about 3 to about 72, from about 3 to about 48, from about 3 to about 24, from about 3 to about 12, from about 3 to about 6, from about 6 to about 120, from about 6 to about 96, from about 6 to about 72, from about 6 to about 48, from about 6 to about 24, from about 6 to about 12, from about 12 to about 120, from about 12 to about 96, from about 12 to about 72, from about 12 to about 48, from about 12 to about 24, from about 24 to about 120, from about 24 to about 96, from about 24 to about 72, from about 24 to about 48, from about 48 to about 120, from about 48 to about 96, from about 48 to about 72, from about 72 to about 120, from about 72 to about 96, or from about 96 to about 120 hours. In certain embodiments, after the transplant of the organ to the recipient, the recipient's blood is passed through the cartridge or contacted with the membrane for about 1 to about 72 hours, about 1 to about 24 hours, about 3 to about 6 hours, about 6 to about 12 hours, about 24 hours, or about 48 hours or about 72 hours. In one embodiment, the recipient's blood is contacted with the membrane or passed through the cartridge from about 6 to about 12 hours. In one embodiment, the recipient's blood is contacted with the membrane or passed through the cartridge from about 3 to about 6 hours. In one embodiment, the recipient's blood is contacted with the membrane or passed through the cartridge for about 12 to about 24 hours. In certain embodiments, after the transplant of the organ to the recipient, the recipient's blood is passed through the cartridge or contacted with the membrane for about 1 hour, about 3 hours, about 6 hours, about 9 hours, about 12 hours, about 24 hours, about 48 hours or about 72 hours.

In certain embodiments, the recipient's blood is passed through the cartridge or contacted with the membrane within about 0.5 to about 120 hours after the transplant of the organ to the recipient. For example, the recipient's blood may be passed through the cartridge within from about 0.5 to about 120, about 0.5 to about 96, from about 0.5 to about 72, from about 0.5 to about 48, from about 0.5 to about 24, from about 0.5 to about 12, from about 0.5 to about 6, from about 0.5 to about 3, from about 0.5 to about 1, from about 1 to about 120, from about 1 to about 96, from about 1 to about 72, from about 1 to about 48, from about 1 to about 24, from about 1 to about 12, from about 1 to about 6, from about 1 to about 3, from about 3 to about 120, from about 3 to about 96, from about 3 to about 72, from about 3 to about 48, from about 3 to about 24, from about 3 to about 12, from about 3 to about 6, from about 6 to about 120, from about 6 to about 96, from about 6 to about 72, from about 6 to about 48, from about 6 to about 24, from about 6 to about 12, from about 12 to about 120, from about 12 to about 96, from about 12 to about 72, from about 12 to about 48, from about 12 to about 24, from about 24 to about 120, from about 24 to about 96, from about 24 to about 72, from about 24 to about 48, from about 48 to about 120, from about 48 to about 96, from about 48 to about 72, from about 72 to about 120, from about 72 to about 96, or from about 96 to about 120 hours after the transplant of the organ to the recipient. In certain embodiments, the recipient's blood is passed through the cartridge or contacted with the membrane within about 12 hours, about 1 day, about 2 days, about 3 days, about 4 days, about 5 days, about 6 days, about 1 week, about 2 weeks, about 3 weeks, about 4 weeks, about 6 weeks, about 8 weeks, about 10 weeks, or about 12 weeks after the transplant of the organ to the recipient.

In certain embodiments, the recipient's blood is passed through the cartridge within 30 days, within 29 days, within 28 days, within 21 days, within 14 days, within 7 days, within 6 days, within 5 days, within 4 days, within 3 days, within 2 days or within 1 day of the recipient receiving the transplant of an organ. For example, the organ is a kidney, a liver, a lung, a heart, a pancreas, or a small intestine.

In certain embodiments, the recipient's blood is passed through the cartridge or contacted with the membrane about 0.5 to about 120 hours before the transplant of the organ to the recipient. For example, the recipient's blood may be passed through the cartridge from about 0.5 to about 120, about 0.5 to about 96, from about 0.5 to about 72, from about 0.5 to about 48, from about 0.5 to about 24, from about 0.5 to about 12, from about 0.5 to about 6, from about 0.5 to about 3, from about 0.5 to about 1, from about 1 to about 120, from about 1 to about 96, from about 1 to about 72, from about 1 to about 48, from about 1 to about 24, from about 1 to about 12, from about 1 to about 6, from about 1 to about 3, from about 3 to about 120, from about 3 to about 96, from about 3 to about 72, from about 3 to about 48, from about 3 to about 24, from about 3 to about 12, from about 3 to about 6, from about 6 to about 120, from about 6 to about 96, from about 6 to about 72, from about 6 to about 48, from about 6 to about 24, from about 6 to about 12, from about 12 to about 120, from about 12 to about 96, from about 12 to about 72, from about 12 to about 48, from about 12 to about 24, from about 24 to about 120, from about 24 to about 96, from about 24 to about 72, from about 24 to about 48, from about 48 to about 120, from about 48 to about 96, from about 48 to about 72, from about 72 to about 120, from about 72 to about 96, or from about 96 to about 120 hours before the transplant of the organ to the recipient. In certain embodiments, the recipient's blood is passed through the cartridge or contacted with the membrane about 12 hours, about 1 day, about 2 days, about 3 days, about 4 days, about 5 days, about 6 days, about 1 week, about 2 weeks, about 3 weeks, or about 4 weeks before the transplant of the organ to the recipient.

In certain embodiments of any of the foregoing methods, the recipient's blood is passed through the cartridge or contacted with the membrane subsequent to the transplant more than once, for example, 1, 2, 3, 4, 5, 6, 7, 8, 9, 10, 11, 12, 14, 16, 18, 20, 22, 24, or more than 24 times. It is understood that an individual cartridge or membrane may be therapeutically effective for a limited amount of time, and as such, after that amount of time an individual cartridge or membrane will have to be replaced. Accordingly, when a recipient's blood is passed through a cartridge or contacted with a membrane more than once, it is contemplated that in each instance the cartridge or membrane may be the same cartridge or membrane or a different cartridge or membrane.

In certain embodiments, the recipient's blood is passed through the cartridge or contacted with the membrane multiples times (e.g., repeatedly, e.g., at regular intervals) over a treatment period subsequent to the transplant. For example, in certain embodiments the recipient's blood is passed through the cartridge or contacted with the membrane about every 12 hours, about every 24 hours, about every 2 days, about every 3 days, about every 4 days, about every 5 days, about every 6 days or about every 7 days, in each instance for about 1 hour, about 2 hours, about 3 hours, about 4 hours, about 5 hours, about 6 hours, about 9 hours, or about 12 hours, about every 3 hours, about every 6 hours, over a treatment period of about 1 day, about 2 days, about 3 days, about 4 days, about 5 days, about 6 days, about 1 week, about 2 weeks, about 3 weeks, about 4 weeks, about 6 weeks, about 8 weeks, about 10 weeks, or about 12 weeks.

In certain embodiments the recipient's blood is passed through the cartridge or contacted with the membrane subsequent to the transplant from about every day to about every 7 days, from about every day to about every 6 days, from about every day to about every 5 days, from about every day to about every 4 days, from about every day to about every 3 days, from about every day to about every 2 days, from about every 2 days to about every 7 days, from about every 2 days to about every 6 days, from about every 2 days to about every 5 days, from about every 2 days to about every 4 days, from about every 2 days to about every 3 days, from about every 3 days to about every 7 days, from about every 3 days to about every 6 days, from about every 3 days to about every 5 days, from about every 3 days to about every 4 days, from about every 4 days to about every 7 days, from about every 4 days to about every 6 days, from about every 4 days to about every 5 days, from about every 5 days to about every 7 days, from about every 5 days to about every 6 days, or from about every 6 days to about every 7 days. In certain embodiments, in each instance the recipient's blood is passed through the cartridge or contacted with the membrane for from about 1 hour to about 2 hours, from about 1 hour to about 3 hours, from about 1 hour to about 4 hours, from about 1 hour to about 5 hours, from about 1 hour to about 6 hours, from about 1 hour to about 9 hours, from about 1 hour to about 12 hours, from about 2 hours to about 3 hours, from about 2 hours to about 4 hours, from about 2 hours to about 5 hours, from about 2 hours to about 6 hours, from about 2 hours to about 9 hours, from about 2 hours to about 12 hours from about 3 hours to about 4 hours, from about 3 hours to about 5 hours, from about 3 hours to about 6 hours, from about 3 hours to about 9 hours, from about 3 hours to about 12 hours, from about 4 hours to about 5 hours, from about 4 hours to about 6 hours, from about 4 hours to about 9 hours, from about 4 hours to about 12 hours, from about 5 hours to about 6 hours, from about 5 hours to about 9 hours, from about 5 hours to about 12 hours, from about 6 hours to about 9 hours, from about 6 hours to about 12 hours, or from about 9 hours to about 12 hours. In certain embodiments, the recipient's blood is passed through the cartridge or contacted over a treatment period of from about 1 day to about 12 weeks, from about 1 day to about 10 weeks, from about 1 days to about 8 weeks, from about 1 day to about 6 weeks, from about 1 day to about 4 weeks, from about 1 day to about 2 weeks, from about 1 day to about 1 week, from about 1 day to about 5 days, from about 1 day to about 3 days, from about 3 days to about 12 weeks, from about 3 days to about 10 weeks, from about 3 days to about 8 weeks, from about 3 days to about 6 weeks, from about 3 days to about 4 weeks, from about 3 days to about 2 weeks, from about 3 days to about 1 week, from about 3 days to about 5 days, from about 5 days to about 12 weeks, from about 5 days to about 10 weeks, from about 5 days to about 8 weeks, from about 5 days to about 6 weeks, from about 5 days to about 4 weeks, from about 5 days to about 2 weeks, from about 5 days to about 1 week, from about 1 week to about 12 weeks, from about 1 week to about 10 weeks, from about 1 week to about 8 weeks, from about 1 week to about 6 weeks, from about 1 week to about 4 weeks, from about 1 week to about 2 weeks, from about 2 weeks to about 12 weeks, from about 2 weeks to about 10 weeks, from about 2 weeks to about 8 weeks, from about 2 weeks to about 6 weeks, from about 2 weeks to about 4 weeks, from about 4 weeks to about 12 weeks, from about 4 weeks to about 10 weeks, from about 4 weeks to about 8 weeks, from about 4 weeks to about 6 weeks, from about 6 weeks to about 12 weeks, from about 6 weeks to about 10 weeks, from about 6 weeks to about 8 weeks, from about 8 weeks to about 12 weeks, from about 8 weeks to about 10 weeks, or from about 10 weeks to about 12 weeks.

In certain embodiments, (i) prior to and/or during the transplant, a cartridge or membrane (e.g, a first cartridge or membrane) is connected to the recipient via an extracorporeal circuit comprising a line from an artery or vein of the recipient and a line to a vein of the recipient, and (ii) after the transplant, a cartridge or membrane (e.g., a second cartridge or membrane) is connected to the recipient via an extracorporeal circuit comprising a line from an artery or vein of the recipient and a line to a vein of the recipient.

The composition of the material making up the blood pump, ultrafiltrate pump, IV fluid return pump, or tubing is preferably a biocompatible material, for example, polyvinylchloride. The tubing may be flexible and have dimensions complementary with associated hemofilter connections, ultrafiltrate recycling device connections, replacement fluid reservoir connection, joints, stop cocks, or pump heads.

In certain embodiments, fluid circuits incorporating the membrane or cartridge optionally can also perform other blood treatments. For example, fluid circuits optionally can further include additional devices that can filter, oxygenate, warm, or otherwise treat the blood before or after the blood enters the cartridge.

In certain embodiments, the membranes, cartridges and/or the fluid circuits incorporating the membranes or cartridges are controlled by a processor (e.g., computer software). In such embodiments, a device can be configured to detect changes within a recipient and provide such information to the processor. In some embodiments, the fluid circuit can automatically process the recipient's blood through the cartridge in response to such information. In other embodiments, a health professional is alerted and initiates treatment.

Exemplary membranes, cartridges, and blood circuits that may be useful in the practice of the invention are disclosed, for example, in U.S. Pat. Nos. 8,597,516 and 6,787,040.

### III. Therapeutic Uses

The methods disclosed herein can be used to improve organ transplant outcomes in a recipient of the transplanted organ. In certain embodiments, the organ or organs is one or more of a heart, lung, kidney, liver, intestine, or pancreas. In one embodiment, the organ is a heart. In another embodiment, the organ is a lung or lungs. In another embodiment, the organ is a kidney. In another embodiment, the organ is a liver. In another embodiment, the organ is intestine. In another embodiment, the organ is a pancreas.

As used herein, the terms “subject” and “recipient” are used interchangeably and refer to an organism that is an organ recipient and is to be treated by the methods and compositions described herein. Such organisms preferably include, but are not limited to, mammals (e.g., murines, primates (e.g., simians), equines, bovines, porcines, canines, felines, and the like), and more preferably includes humans. In a preferred embodiment, the recipient is a human recipient. Recipients may include both adult and pediatric recipients.

In certain embodiments, the methods disclosed herein improve a function of a transplanted organ in a recipient of the transplanted organ. For example, in one embodiment, the transplanted organ is a heart. In another embodiment, the transplanted organ is a liver. In another embodiment, the transplanted organ is a kidney. In another embodiment, the transplanted organ is a lung. In another embodiment, the transplanted organ is an intestine. In another embodiment, the transplanted organ is a pancreas. In another embodiment, the transplanted organ is not a liver. In another embodiment, the transplanted organ is not a kidney. In another embodiment, the transplanted organ is not a heart. In another embodiment, the transplanted organ is not a lung. In another embodiment, the transplanted organ is not a pancreas. In another embodiment, the transplanted organ is not an intestine. In certain embodiments, the methods disclosed herein improve a function of one or more transplanted organs in a recipient of the one or more transplanted organs. For example, in certain embodiments, the one or more transplanted organs are (i) a heart and/or a lung, (ii) a heart and/or a kidney, (iii) a heart and/or a liver, (iv) a lung and/or a kidney, (v) a lung and/or a liver, (vi) a kidney and/or a liver, (vii) a heart, a lung, and/or a kidney, (viii) a heart, a lung, and/or a liver, (ix) a lung, a kidney, and/or a liver, or (x) a heart, a lung, a kidney, and/or a liver.

In certain embodiments, the methods disclosed herein treat or prevent primary graft dysfunction of a transplanted organ in a recipient of the transplanted organ. For example, in one embodiment, the transplanted organ is a heart (See, e.g., Kobashigawa et al. (2014) J. HEART LUNG TRANSPLANT, 33(4):327-40, and Nicoara et al. (2018) AM J. TRANSPLANT, 18(6):1461-1470). In another embodiment, the transplanted organ is a liver. (See, e.g., Chen et al. (2014) HEPATOBILIARY PANCREAT DIS INT, 13(2):125-37). In another embodiment, the transplanted organ is a kidney (See, e.g., Foster et al. AM J. KIDNEY DIS, 44(2):376-81). In another embodiment, the transplanted organ is a lung (See, e.g., Suzuki et al. (2013) SEMIN RESPIR CRIT CARE MED, 34(3): 305-319). In another embodiment, the transplanted organ is an intestine. In another embodiment, the transplanted organ is a pancreas. In another embodiment, the transplanted organ is not a liver. In another embodiment, the transplanted organ is not a kidney. In another embodiment, the transplanted organ is not a heart. In another embodiment, the transplanted organ is not a lung. In another embodiment, the transplanted organ is not a pancreas. In another embodiment, the transplanted organ is not an intestine. In certain embodiments, the methods disclosed herein treat or prevent primary graft dysfunction of one or more transplanted organs in a recipient of the transplanted organs. For example, in certain embodiments, the one or more organs are (i) a heart and/or a lung, (ii) a heart and/or a kidney, (iii) a heart and/or a liver, (iv) a lung and/or a kidney, (v) a lung and/or a liver, (vi) a kidney and/or a liver, (vii) a heart, a lung, and/or a kidney, (viii) a heart, a lung, and/or a liver, (ix) a lung, a kidney, and/or a liver, or (x) a heart, a lung, a kidney, and/or a liver.

In certain embodiments, the methods disclosed herein treat or prevent acute rejection of a transplanted organ in a recipient of the transplanted organ. Acute rejection generally occurs within the first several months after transplant, i.e., within the first year following transplant For example, in one embodiment, the transplanted organ is a heart. In another embodiment, the transplanted organ is a liver. In another embodiment, the transplanted organ is a kidney. In another embodiment, the transplanted organ is a lung. In another embodiment, the transplanted organ is an intestine. In another embodiment, the transplanted organ is a pancreas. In another embodiment, the transplanted organ is not a liver. In another embodiment, the transplanted organ is not a kidney. In another embodiment, the transplanted organ is not a heart. In another embodiment, the transplanted organ is not a lung. In another embodiment, the transplanted organ is not a pancreas. In another embodiment, the transplanted organ is not an intestine. In certain embodiments, the methods disclosed herein treat or prevent acute rejection of one or more transplanted organs in a recipient of the transplanted organs. For example, in certain embodiments, the one or more organs are (i) a heart and/or a lung, (ii) a heart and/or a kidney, (iii) a heart and/or a liver, (iv) a lung and/or a kidney, (v) a lung and/or a liver, (vi) a kidney and/or a liver, (vii) a heart, a lung, and/or a kidney, (viii) a heart, a lung, and/or a liver, (ix) a lung, a kidney, and/or a liver, or (x) a heart, a lung, a kidney, and/or a liver.

In certain embodiments, the methods disclosed herein reduce or prevent delayed graft function of a transplanted organ in a recipient of the transplanted organ. For example, in one embodiment, the transplanted organ is a heart. In another embodiment, the transplanted organ is a liver. In another embodiment, the transplanted organ is a kidney (See, e.g., Siedlecki et al. (2012) AM J. TRANSPLANT, 11(11): 2279-2296. In another embodiment, the transplanted organ is a lung. In another embodiment, the transplanted organ is an intestine. In another embodiment, the transplanted organ is a pancreas. In another embodiment, the transplanted organ is not a liver. In another embodiment, the transplanted organ is not a kidney. In another embodiment, the transplanted organ is not a heart. In another embodiment, the transplanted organ is not a lung. In another embodiment, the transplanted organ is not a pancreas. In another embodiment, the transplanted organ is not an intestine. In certain embodiments, the methods disclosed herein reduce or prevent delayed graft function of one or more transplanted organs in a recipient of the transplanted organs. For example, in certain embodiments, the one or more organs are (i) a heart and/or a lung, (ii) a heart and/or a kidney, (iii) a heart and/or a liver, (iv) a lung and/or a kidney, (v) a lung and/or a liver, (vi) a kidney and/or a liver, (vii) a heart, a lung, and/or a kidney, (viii) a heart, a lung, and/or a liver, (ix) a lung, a kidney, and/or a liver, or (x) a heart, a lung, a kidney, and/or a liver.

In certain embodiments, the methods disclosed herein remove inflammatory molecules from the blood of a recipient of a transplanted organ. The removal can occur before, during, or after the transplant. For example, in one embodiment, the transplanted organ is a heart. In another embodiment, the transplanted organ is a liver. In another embodiment, the transplanted organ is a kidney. In another embodiment, the transplanted organ is a lung. In another embodiment, the transplanted organ is an intestine. In another embodiment, the transplanted organ is a pancreas. In another embodiment, the transplanted organ is not a liver. In another embodiment, the transplanted organ is not a kidney. In another embodiment, the transplanted organ is not a heart. In another embodiment, the transplanted organ is not a lung. In another embodiment, the transplanted organ is not a pancreas. In another embodiment, the transplanted organ is not an intestine. In certain embodiments, the methods disclosed herein remove inflammatory molecules from the blood of a recipient of one or more transplanted organs. For example, in certain embodiments, the one or more organs are (i) a heart and/or a lung, (ii) a heart and/or a kidney, (iii) a heart and/or a liver, (iv) a lung and/or a kidney, (v) a lung and/or a liver, (vi) a kidney and/or a liver, (vii) a heart, a lung, and/or a kidney, (viii) a heart, a lung, and/or a liver, (ix) a lung, a kidney, and/or a liver, or (x) a heart, a lung, a kidney, and/or a liver.

In certain embodiments, the methods disclosed herein achieve a clinical endpoint indicative of a successful organ transplant in a recipient of a transplanted organ. For example, in one embodiment, the transplanted organ is a heart. In another embodiment, the transplanted organ is a liver. In another embodiment, the transplanted organ is a kidney. In another embodiment, the transplanted organ is a lung. In another embodiment, the transplanted organ is an intestine. In another embodiment, the transplanted organ is a pancreas. In another embodiment, the transplanted organ is not a liver. In another embodiment, the transplanted organ is not a kidney. In another embodiment, the transplanted organ is not a heart. In another embodiment, the transplanted organ is not a lung. In another embodiment, the transplanted organ is not a pancreas. In another embodiment, the transplanted organ is not an intestine. In certain embodiments, the methods disclosed herein achieve a clinical endpoint indicative of one or more successful organ transplants treat in a recipient of the one or more transplanted organs. For example, in certain embodiments, the one or more organs are (i) a heart and/or a lung, (ii) a heart and/or a kidney, (iii) a heart and/or a liver, (iv) a lung and/or a kidney, (v) a lung and/or a liver, (vi) a kidney and/or a liver, (vii) a heart, a lung, and/or a kidney, (viii) a heart, a lung, and/or a liver, (ix) a lung, a kidney, and/or a liver, or (x) a heart, a lung, a kidney, and/or a liver.

For example, in certain embodiments, the method results in the transplant recipient meeting one or more clinical end points indicative of a successful organ transplant after their blood has been treated according to the methods of the invention before, during, and/or after organ transplant surgery.

In certain embodiments, the methods of the invention result in a subject achieving one or more clinical end points indicative of a successful organ transplant more quickly than a subject not receiving treatment according to the methods of the invention.

Clinical endpoints indicative of a successful transplant of a transplanted heart include, for example: left ventricular ejection fraction (LVEF) stabilized at >40% (as measured by echocardiography); right arterial pressure (RAP) stabilized at <15 mm Hg; pulmonary capillary wedge pressure (PCWP) stabilized at <20 mm Hg; cardiac index (CI) stabilized at >2.0 L/min/m²; inotrope is not required or required only at low doses with an inotrope score <10 (inotrope score=dopamine×1+dobutamine×1+amrinone×1+milrinone×15+epinephrine×100+norepinephrine×100, with each drug dosed in μg/kg/minute); mean arterial pressure (MAP) stabilized at ≥70 mm Hg; no need for an intraaortic balloon pump (IABP), extracorporeal membrane oxygenation (ECMO), left ventricular assist device (LVAD), or biventricular assist or device (BiVAD); right arterial pressure (RAP) stabilized at <15 mm Hg; and no need for right ventricular assist device (RVAD). See, e.g., Kobashigawa et al. (2014) J. HEART LUNG TRANSPLANT, 33(4):327-40. Additional clinical endpoints indicative of a successful transplant of a transplanted heart include, for example: lack of delayed graft function; and lack of acute humoral or cellular rejection on endomyocardial biopsy done either for recipient clinical indication or as scheduled evaluation of the graft. See, e.g., Stewart et al. (2005) J. HEART LUNG TRANSPLANT, 24:1710-20. Additional clinical endpoints indicative of a successful transplant of a transplanted heart include, for example: more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and a more rapid return to normal daily activities (relative to a recipient who has not been treated with a method of the invention). In certain embodiments, a recipient of a heart transplant treated with a method of the invention exhibits one, two, three, four, five, or more than five of the aforementioned endpoints.

Clinical endpoints indicative of a successful transplant of a transplanted lung include, for example: PaO₂/FiO₂ (partial pressure or arterial oxygen/percentage of inspired oxygen) stabilized at greater than 300; liberation from supplemental oxygen; and lack of pathology on chest radiographs (or mild pathology which rapidly resolves). See, e.g., Suzuki et al. (2013) SEMIN RESPIR CRIT CARE MED, 34(3): 305-319. Additional clinical endpoints indicative of a successful transplant of a transplanted lung include, for example: lack of delayed graft function; lack of humoral or cellular rejection on transbronchial, or other biopsy done either for recipient clinical indication or as scheduled evaluation of the graft. See, e.g., Parulekar et al. (2019) J. THORAC DIS, 11(Suppl 14): S1732-39. Additional clinical endpoints indicative of a successful transplant of a transplanted lung include, for example: more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and a more rapid return to normal daily activities (relative to a recipient who has not been treated with a method of the invention). In certain embodiments, a recipient of a lung transplant treated with a method of the invention exhibits one, two, three, four, five, or more than five of the aforementioned endpoints.

Clinical endpoints indicative of a successful transplant of a transplanted kidney include, for example: if placed on machine perfusion, low or declining resistive index to flow of perfusion fluid; increased or normalized glomerular filtration rate, as indicated by creatinine clearance rising or serum creatinine levels and blood urea nitrogen levels declining toward normal; and lack of or resolved albuminuria. Additional clinical endpoints indicative of a successful transplant of a transplanted kidney include, for example: no requirement for renal replacement treatment in the first week after transplant; lack of acute humoral or cellular rejection on renal biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and a more rapid return to normal daily activities (relative to a recipient who has not been treated with a method of the invention). In certain embodiments, a recipient of a kidney transplant treated with a method of the invention exhibits one, two, three, four, five, or more than five of the aforementioned endpoints.

Clinical endpoints indicative of a successful transplant of a transplanted liver include, for example: on postoperative day 7 a bilirubin level of less than 10 mg/dL; an international normalized ratio of less than 1.6; and alanine aminotransferase or aspartate aminotransferase levels not greater than 2000 IU/L within 7 postoperative days. See, e.g., Olthoff et al. (2010) LIVER TRANSPL, 16:943-49. Additional clinical endpoints indicative of a successful transplant of a transplanted liver include, for example: if low serum sodium, an increase in serum sodium to ≥135 mEq/Liter; and if high serum sodium, a decrease in serum sodium to ≤145 mEq/Liter. See, e.g., Biggins (2006) GASTROENTEROLOGY, 130:1652-60. Additional clinical endpoints indicative of a successful transplant of a transplanted liver include, for example: if elevated serum creatinine, a reduction or normalization of serum creatinine. See, e.g., Barri (2009) LIVER TRANSPLANTATION, 15:475-83. Additional clinical endpoints indicative of a successful transplant of a transplanted liver include, for example: lack of early allograph dysfunction; lack of acute humoral or cellular rejection on biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and a more rapid return to normal daily activities (relative to a recipient who has not been treated with a method of the invention). See, e.g., Sudan (2014) AMERICAN JOURNAL OF TRANSPLANTATION, 14: 1976-84. In certain embodiments, a recipient of a liver transplant treated with a method of the invention exhibits one, two, three, four, five, or more than five of the aforementioned endpoints.

Clinical endpoints indicative of a successful transplant of a transplanted intestine include, for example: lack of early graft dysfunction; lack of acute humoral or cellular rejection on biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); less renal dysfunction (relative to a recipient who has not been treated with a method of the invention); and a more rapid return to normal daily activities (relative to a recipient who has not been treated with a method of the invention). In certain embodiments, a recipient of an intestine transplant treated with a method of the invention exhibits one, two, three, four, five, or more than five of the aforementioned endpoints.

Clinical endpoints indicative of a successful transplant of a transplanted pancreas include, for example: lack of early graft dysfunction, lack of acute humoral or cellular rejection on biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); less multiple organ failure (relative to a recipient who has not been treated with a method of the invention); less renal dysfunction (relative to a recipient who has not been treated with a method of the invention); and a more rapid return to normal daily activities (relative to a recipient who has not been treated with a method of the invention). See, e.g., Gruessner et al. (2004) AMERICAN J. TRANSPL., 4: 2018-26. In certain embodiments, a recipient of a pancreas transplant treated with a method of the invention exhibits one, two, three, four, five, or more than five of the aforementioned endpoints.

The methods described herein may reduce systemic inflammation in a recipient. For example, methods and cartridges described herein may reduce a level of an inflammatory molecule in a recipient, e.g., in a body fluid (e.g., blood, plasma, serum, or urine), tissue and/or cell in a recipient, e.g., by at least about 1%, about 5%, about 10%, about 20%, about 30%, about 40%, about 50%, about 60%, about 70%, about 80%, about 90%, about 95%, or more, relative to levels in an untreated or control subject. For example, methods and cartridges described herein may reduce a level of an inflammatory cytokine or chemokine. Exemplary inflammatory cytokines or chemokines include IL-1-β, IL-4, IL-5, IL-6, IL-8, IL-12, IL-13, IL-17, IL-21, IL-22, IL-23, IL-27, IFN, CCL-2, CCL-3, CCL-5, CCL-20, CXCL-5, CXCL-10, CXCL-12, CXCL-13, and TNF-α. Additional exemplary inflammatory molecules include MCP-1, IP-10, C3a, C5a, soluble TNF receptor II, soluble TNF receptor I, matrix metalloproteinase-9, matrix metalloproteinase-7, IL-10, soluble gp130, lipopolysaccharide, and procalcitonin. In certain embodiments, the inflammatory cytokines or chemokines that maybe removed from the blood of the recipient can include one or more of IL-6, TNF-α C3a, and C5a. It is understood that reference to an inflammatory molecule includes the inflammatory molecule in both an unbound state or in complex with a corresponding ligand. Exemplary inflammatory molecule-ligand complexes include an IL-6/IL-6 soluble receptor complex, a TNF-α/soluble TNF receptor complex, and an albumin/inflammatory cytokine complex.

The methods described herein can be used alone or in combination with other therapeutic agents and/or modalities. The term administered “in combination,” as used herein, is understood to mean that two (or more) different treatments are delivered to the subject, such that the effects of the treatments on the subject overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment improves an outcome to a greater extent, than would be seen if the second treatment were administered in the absence of the first treatment, or the analogous situation is seen with the first treatment. In certain embodiments, delivery is such that improvement of an outcome is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a disclosed method is administered in combination with an anticoagulant, for example, heparin, a citrate salt, etc. Anticoagulation protocols, such as systemic heparin or regional citrate, are currently established and routinely used in clinical hemofiltration. Additional exemplary anticoagulants include warfarin, FXa inhibitors (e.g., enoxaparin, rivaroxaban, apixaban, betrixaban and edoxaban), thrombin inhibitors (e.g., hirudin, lepirudin, bivalirudin, argatroban and dabigatran), and coumarins.

Throughout the description, where devices or compositions are described as having, including, or comprising specific components, or where processes and methods are described as having, including, or comprising specific steps, it is contemplated that, additionally, there are devices or compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a device, a composition, or a method described herein can be combined in a variety of ways without departing from the spirit and scope of the present invention, whether explicit or implicit herein. For example, where reference is made to a particular device, that device can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remains operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This Example describes the treatment of a heart transplant recipient with a hemofilter during and after organ transplant.

Recipients of a transplanted heart are treated for 6 to 12 hours beginning during the transplant surgery and continuing after surgery with a CLR 2.0 Hemofilter (Seastar Medical, Inc., Denver, Colo.). The filtrate-substitution fluid exchange dose is 35 ml/kg/hour. Substitution fluid is delivered pre-dilution (upstream of the hemofilter). Fluid removal is as indicated by the recipients's hydration status. Vascular access is, for example, by a size 13.5 to 14.5 French scale (Fr) dialysis catheter in the right internal jugular vein, left internal jugular vein, and either femoral vein. Recipients receive no anticoagulation therapy. Together, these results demonstrate that treatment of a heart transplant recipient with a hemofilter, e.g., a CLR 2.0 hemofilter, improves heart transplant outcomes.

It is contemplated that treatment reduces or eliminates heart graft inflammation originating in the donor and aggravated by the ischemic period, or will reduce or eliminate the need for mechanical ventilation and cardiovascular support drugs. It is also contemplated that myocardial injury associated with donor inflammation will be abated hastening liberation from cardiac support drugs, cardiac support devices, and mechanical ventilation. Furthermore, it is contemplated that reduced need for cardiac and pulmonary support will hasten the recipients liberation from intensive care and shorten hospitalization. It is also contemplated that the abatement of inflammation will persist after CLR 2.0 hemofiltration, reducing the risk of infection and hastening recipient recovery and mobilization.

Additionally, it is contemplated that recipients may exhibit: PaO₂/FiO₂ stabilized at >300; liberation from supplemental oxygen; lack of pathology on chest radiographs (or mild pathology which rapidly resolves); lack of delayed graft function; lack of humoral or cellular rejection on myocardial or endocardial biopsy, or other biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and/or more rapid return to normal daily activities (relative to a recipient who has not been treated with a method of the invention).

Such outcomes demonstrate that treatment of a heart transplant recipient with a hemofilter, e.g., a CLR 2.0 hemofilter, will improve heart transplant outcomes.

### Example 2

This Example describes the treatment of a lung transplant recipient with a hemofilter during and after organ transplant.

Recipients of a transplanted lung are treated for 6 to 12 hours beginning during the transplant surgery and continuing after surgery with a CLR 2.0 Hemofilter (Seastar Medical, Inc., Denver, Colo.). The filtrate-substitution fluid exchange dose is 35 ml/kg/hour. Substitution fluid is delivered pre-dilution (upstream of the hemofilter). Fluid removal is as indicated by the recipients's hydration status. Vascular access is, for example, by a size 13.5 to 14.5 French scale (Fr) dialysis catheter in the right internal jugular vein, left internal jugular vein, and either femoral vein. Recipients receive no anticoagulation therapy.

It is contemplated that treatment will reduce or eliminate lung graft inflammation originating in the donor and aggravated by the ischemic period or eliminate the need for mechanical ventilation and cardiovascular support drugs. It is also contemplated that acute lung injury associated with donor inflammation will be abated, hastening liberation from mechanical ventilation. Furthermore, it is contemplated that reduced need for cardiac and pulmonary support will hasten recipient liberation from intensive care and shorten hospitalization. It is also contemplated that abatement of inflammation will persist after CLR 2.0 hemofiltration, reducing the risk of infection and hastening recipient recovery and mobilization.

Additionally, it is contemplated that recipients may exhibit or experience: PaO₂/FiO₂ stabilized at >300; liberation from supplemental oxygen; lack of pathology on chest radiographs (or mild pathology which rapidly resolves); lack of delayed graft function; lack of humoral or cellular rejection on transbronchial, or other biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and/or more rapid return to normal daily activities (relative to a recipient who has not been treated with a method of the invention).

Such outcomes demonstrate that treatment of a lung transplant recipient with a hemofilter, e.g., a CLR 2.0 hemofilter, will improve lung transplant outcomes.

### Example 3

This Example describes the treatment of a kidney transplant recipient with a hemofilter during and after organ transplant.

Recipients of a transplanted kidney are treated for 6 to 12 hours beginning during the transplant surgery and continuing after surgery with a CLR 2.0 Hemofilter (Seastar Medical, Inc., Denver, Colo.). The filtrate-substitution fluid exchange dose is 35 ml/kg/hour. Substitution fluid is delivered pre-dilution (upstream of the hemofilter). Fluid removal is as indicated by the recipients's hydration status. Vascular access is, for example, by a size 13.5 to 14.5 French scale (Fr) dialysis catheter in the right internal jugular vein, left internal jugular vein, and either femoral vein. Recipients receive no anticoagulation therapy.

It is contemplated that treatment will reduce or eliminate kidney graft inflammation originating in the donor and aggravated by the ischemic period, or will reduce or eliminate the need for renal replacement therapy, and cardiac, pulmonary and other renal support, hastening recipient liberation from intensive care and shortening hospitalization. It is contemplated that abatement of inflammation will persist after CLR 2.0 hemofiltration, reducing the risk of infection and hastening recipient recovery and mobilization.

Additionally, it is contemplated that recipients may exhibit or experience: if placed on machine perfusion, low or declining resistive index to flow of perfusion fluid; increased or normalized glomerular filtration rate, as indicated by creatinine clearance rising or serum creatinine levels and blood urea nitrogen levels declining toward normal; lack of or resolved albuminuria; no requirement for renal replacement treatment in the first week after transplant; lack of acute humoral or cellular rejection on renal biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and/or more rapid return to normal daily activities (relative to a recipient who has not been treated with a method of the invention).

It is contemplated that abatement of inflammation persists after CLR 2.0 hemofiltration, reducing the risk of infection and hastening recipient recovery and mobilization.

Such outcomes demonstrate that treatment of a kidney transplant recipient with a hemofilter, e.g., a CLR 2.0 hemofilter, will improve kidney transplant outcomes.

### Example 4

This Example describes the treatment of a liver transplant recipient with a hemofilter during and after organ transplant.

Recipients of transplanted livers are treated for 6 to 12 hours beginning during the transplant surgery and continuing after surgery with a CLR 2.0 Hemofilter (Seastar Medical, Inc., Cardiff-by-the-Sea, Calif.). The filtrate-substitution fluid exchange dose is 35 ml/kg/hour. Substitution fluid is delivered pre-dilution (upstream of the hemofilter). Fluid removal is as indicated by the recipients's hydration status. Vascular access is, for example, by a size 13.5 to 14.5 French scale (Fr) dialysis catheter in the right internal jugular vein, left internal jugular vein, and either femoral vein. Recipients receive no anticoagulation therapy.

It is contemplated that treatment will reduce or eliminate liver graft inflammation originating in the donor and aggravated by the ischemic period and will reduce or eliminate the need for critical supportive therapies, cardiac, pulmonary, renal and other critical support measures, hastening recipient liberation from intensive care and shortening hospitalization. It is contemplated that abatement of inflammation will persist after CLR 2.0 hemofiltration, reducing the risk of infection and hastening recipient recovery and mobilization.

Additionally, it is contemplated that recipients may experience or exhibit: on postoperative day 7 bilirubin less than 10 mg/dL; international normalized ratio less than 1.6; and alanine aminotransferase or aspartate aminotransferase not >2000 IU/L within 7 postoperative days; if low serum sodium an increase in serum sodium to ≥135 mEq/Liter; if high serum sodium a decrease in serum sodium to ≤145 mEq/Liter; if elevated serum creatinine a reduction or normalization of serum creatinine; lack of early allograph dysfunction; lack of acute humoral or cellular rejection on biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); and/or more rapid return to their normal daily activities (relative to a recipient who has not been treated with a method of the invention).

Such outcomes demonstrate that treatment of a liver transplant recipient with a hemofilter, e.g., a CLR 2.0 hemofilter, will improve liver transplant outcomes.

### Example 5

This Example describes the treatment of an intestine transplant recipient with a hemofilter during and after organ transplant.

Recipients of intestine transplants were treated for 6 to 12 hours beginning during the transplant surgery and continuing after surgery with a CLR 2.0 Hemofilter (Seastar Medical, Inc., Denver, Colo.). The filtrate-substitution fluid exchange dose is 35 ml/kg/hour. Substitution fluid is delivered pre-dilution (upstream of the hemofilter). Fluid removal is as indicated by the recipients's hydration status. Vascular access is, for example, by a size 13.5 to 14.5 French scale (Fr) dialysis catheter in the right internal jugular vein, left internal jugular vein, and either femoral vein. Recipients receive no anticoagulation therapy.

It is contemplated that treatment will reduce or eliminate intestinal graft inflammation originating in the donor and aggravated by the ischemic period and will reduce or eliminate the need for cardiac, pulmonary, renal and other critical support measures which will hasten recipient liberation from intensive care and shorten hospitalization. It is also contemplated that the abatement of inflammation will persist after CLR 2.0 hemofiltration, reducing the risk of infection and hastening recipient recovery and mobilization.

Additionally, it is contemplated that recipients may exhibit or experience: lack of early graft dysfunction; lack of acute humoral or cellular rejection on biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); less renal dysfunction (relative to a recipient who has not been treated with a method of the invention); and/or more rapid return to normal daily activities (relative to a recipient who has not been treated with a method of the invention).

Such outcomes demonstrate that treatment of an intestine transplant recipient with a hemofilter, e.g., a CLR 2.0 hemofilter, will improve intestine transplant outcomes.

### Example 6

This Example describes the treatment of a pancreas transplant recipient with a hemofilter during and after organ transplant.

Recipients of pancreas transplants are treated for 6 to 12 hours beginning during the transplant surgery and continuing after surgery with a CLR 2.0 Hemofilter (Seastar Medical, Inc., Denver, Colo.). The filtrate-substitution fluid exchange dose is 35 ml/kg/hour. Substitution fluid is delivered pre-dilution (upstream of the hemofilter). Fluid removal is as indicated by the recipients's hydration status. Vascular access is, for example, by a size 13.5 to 14.5 French scale (Fr) dialysis catheter in the right internal jugular vein, left internal jugular vein, and either femoral vein. Recipients received no anticoagulation therapy.

It is contemplated that treatment will reduce or eliminate intestinal graft inflammation originating in the donor and aggravated by the ischemic period. It is contemplated that treatment will preserve beta cell function and insulin independence, and reduce or eliminate the need for cardiac, pulmonary, renal and other critical support measures, hastening recipient liberation from intensive care and shortening hospitalization. It is contemplated that abatement of inflammation will persist after CLR 2.0 hemofiltration, reducing the risk of infection and hastening recipient recovery and mobilization.

Additionally, it is contemplated that recipients may experience or exhibit: lack of early graft dysfunction, lack of acute humoral or cellular rejection on biopsy done either for recipient clinical indication or as scheduled evaluation of the graft; more rapid post-operative mobilization from mechanical ventilation and inotrope support (relative to a recipient who has not been treated with a method of the invention); fewer intensive care unit and hospital days (relative to a recipient who has not been treated with a method of the invention); fewer infections (relative to a recipient who has not been treated with a method of the invention); less multiple organ failure (relative to a recipient who has not been treated with a method of the invention); less renal dysfunction (relative to a recipient who has not been treated with a method of the invention); and/or more rapid return to their normal daily activities (relative to a recipient who has not been treated with a method of the invention).

Such outcomes demonstrate that treatment of a pancreas transplant recipients with a hemofilter, e.g., a CLR 2.0 hemofilter, will improve pancreas transplant outcomes.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of improving a function of a transplanted organ in a recipient of the transplanted organ, the method comprising contacting blood from the recipient with an extracorporeal membrane defining a plurality of pores having an average pore size of at least 60 kDa to permit inflammatory molecules in the blood to pass through the pores for removal from the blood, whereupon the blood depleted of the inflammatory molecules is returned to the recipient.

2. A method of treating or preventing primary graft dysfunction of a transplanted organ in a recipient of the transplanted organ, the method comprising contacting blood from the recipient with an extracorporeal membrane defining a plurality of pores having an average pore size of at least 60 kDa to permit inflammatory molecules in the blood to pass through the pores for removal from the blood, whereupon the blood depleted of the inflammatory molecules is returned to the recipient.

3. A method of treating or preventing acute rejection of a transplanted organ by a recipient of the transplanted organ, the method comprising contacting blood from the recipient with an extracorporeal membrane defining a plurality of pores having an average pore size of at least 60 kDa to permit inflammatory molecules in the blood to pass through the pores for removal from the blood, whereupon the blood depleted of the inflammatory molecules is returned to the recipient.

4. A method of treating or preventing delayed graft function of a transplanted organ in a recipient of the transplanted organ, the method comprising contacting blood from the recipient with an extracorporeal membrane defining a plurality of pores having an average pore size of at least 60 kDa to permit inflammatory molecules in the blood to pass through the pores for removal from the blood, whereupon the blood depleted of the inflammatory molecules is returned to the recipient.

5. A method of achieving a clinical endpoint indicative of a successful organ transplant in a recipient of a transplanted organ, the method comprising contacting blood from the recipient with an extracorporeal membrane defining a plurality of pores having an average pore size of at least 60 kDa to permit inflammatory molecules in the blood to pass through the pores for removal from the blood, whereupon the blood depleted of the inflammatory molecules is returned to the recipient.

6. A method of removing inflammatory molecules from the blood of a recipient of a transplanted organ, the method comprising contacting blood from the recipient with an extracorporeal membrane defining a plurality of pores having an average pore size of at least 60 kDa to permit inflammatory molecules in the blood to pass through the pores for removal from the blood, whereupon the blood depleted of the inflammatory molecules is returned to the recipient.

7. The method of any one of claims 1-6, wherein the blood from the recipient is contacted with the extracorporeal membrane during and/or after the transplant of the organ to the recipient.

8. The method of any one of claims 1-7, wherein the pores are defined by a wall of a semi-permeable hollow fiber.

9. A method of improving a function of a transplanted organ in a recipient of the transplanted organ, the method comprising passing blood from the recipient through an extracorporeal cartridge comprising a housing and a plurality of semi-permeable hollow fibers disposed therein, each of the semi-permeable hollow fibers defining a lumen and a plurality of pores, such that when the blood traverses the lumens of the hollow fibers, inflammatory molecules from the blood pass through the pores and are removed from the blood, whereupon the blood depleted of inflammatory molecules is returned to the recipient, wherein the blood from the recipient is passed through the cartridge during and/or after the transplant of the organ to the recipient.

10. A method of treating or preventing primary graft dysfunction of a transplanted organ in a recipient of the transplanted organ, the method comprising passing blood from the recipient through an extracorporeal cartridge comprising a housing and a plurality of semi-permeable hollow fibers disposed therein, each of the semi-permeable hollow fibers defining a lumen and a plurality of pores, such that when the blood traverses the lumens of the hollow fibers, inflammatory molecules from the blood pass through the pores and are removed from the blood, whereupon the blood depleted of inflammatory molecules is returned to the recipient, wherein the blood from the recipient is passed through the cartridge during and/or after the transplant of the organ to the recipient.

11. A method of treating or preventing acute rejection of a transplanted organ by a recipient of the transplanted organ, the method comprising passing blood from the recipient through an extracorporeal cartridge comprising a housing and a plurality of semi-permeable hollow fibers disposed therein, each of the semi-permeable hollow fibers defining a lumen and a plurality of pores, such that when the blood traverses the lumens of the hollow fibers, inflammatory molecules from the blood pass through the pores and are removed from the blood, whereupon the blood depleted of inflammatory molecules is returned to the recipient, wherein the blood from the recipient is passed through the cartridge during and/or after the transplant of the organ to the recipient.

12. A method of treating or preventing delayed graft function of a transplanted organ in a recipient of the transplanted organ, the method comprising passing blood from the recipient through an extracorporeal cartridge comprising a housing and a plurality of semi-permeable hollow fibers disposed therein, each of the semi-permeable hollow fibers defining a lumen and a plurality of pores, such that when the blood traverses the lumens of the hollow fibers, inflammatory molecules from the blood pass through the pores and are removed from the blood, whereupon the blood depleted of inflammatory molecules is returned to the recipient, wherein the blood from the recipient is passed through the cartridge during and/or after the transplant of the organ to the recipient.

13. A method of achieving a clinical endpoint indicative of a successful organ transplant in a recipient of a transplanted organ, the method comprising passing blood from the recipient through an extracorporeal cartridge comprising a housing and a plurality of semi-permeable hollow fibers disposed therein, each of the semi-permeable hollow fibers defining a lumen and a plurality of pores, such that when the blood traverses the lumens of the hollow fibers, inflammatory molecules from the blood pass through the pores and are removed from the blood, whereupon the blood depleted of inflammatory molecules is returned to the recipient, wherein the blood from the recipient is passed through the cartridge during and/or after the transplant of the organ to the recipient.

14. A method of removing inflammatory molecules from the blood of a recipient of a transplanted organ, the method comprising passing blood from the recipient through an extracorporeal cartridge comprising a housing and a plurality of semi-permeable hollow fibers disposed therein, each of the semi-permeable hollow fibers defining a lumen and a plurality of pores, such that when the blood traverses the lumens of the hollow fibers, inflammatory molecules from the blood pass through the pores and are removed from the blood, whereupon the blood depleted of inflammatory molecules is returned to the recipient, wherein the blood from the recipient is passed through the cartridge during and/or after the transplant of the organ to the recipient.

15. The method of any one of claims 1-14, wherein the pores have an average pore size of from about 60 kDa to about 150 kDa.

16. The method of any one of claims 1-14, wherein the pores have an average pore size of greater than 65 kDa.

17. The method of any one of claims 1-14, wherein the pores have an average pore size of no greater than 65 kDa.

18. The method of any one of claims 1-14, wherein the pores have an average pore size from about 60 kDa to about 65 kDa.

19. The method of any one of claims 1-18, wherein the recipient is human.

20. The method of any one of claims 1-19, wherein the recipient is an adult human recipient.

21. The method of any one of claims 1-18, wherein the recipient is a pediatric human recipient.

22. The method of any one of claims 8-21, wherein the hollow fibers comprise a polymer.

23. The method of any one of claims 8-21, wherein the hollow fibers comprise polysulfone.

24. The method of any one of claims 8-23, wherein the lumens of the hollow fibers have a diameter from about 100 μM to about 700 μM.

25. The method of claim 24, wherein the lumens have a diameter from about 175 μM to about 225 μM.

26. The method of claim 24, wherein the lumens have a diameter from about 600 μM to about 700 μM.

27. The method of any one of claims 8-26, wherein the surface area of the hollow fibers is from about 0.01 m² to about 4.0 m².

28. The method of claim 27, wherein the surface area of the hollow fibers is from about 1.9 m² to about 2.1 m².

29. The method of claim 27, wherein the surface area of the hollow fibers is from about 0.05 m² to about 0.1 m², or from about 0.25 m² to about 0.75 m², or from about 1.0 m² to about 1.5 m².

30. The method of any of claims 9-29, wherein the cartridge comprises a fluid inlet port and a fluid outlet port.

31. The method of any of claims 9-30, wherein the cartridge comprises one or more ultrafiltrate ports.

32. The method of any one of claims 1-31, wherein the inflammatory molecules removed from the blood are selected from one or more of IL-4, IL-6, IL-8, TNF-α, IL-1β, MCP-1, CCL2, IP-10, CXCL10, C3a, C5a, soluble TNF receptor II, soluble TNF receptor I, matrix metalloproteinase-9, matrix metalloproteinase-7, IL-10, soluble gp130, lipopolysaccharide (LPS), or procalcitonin.

33. The method of any one of claims 9-32 wherein the flow rate of blood through the cartridge is from about 100 mL/min to about 600 mL/min.

34. The method of any one of claims 9-32, wherein the flow rate of blood through the cartridge is from about 100 mL/min to about 400 mL/min.

35. The method of any one of claims 9-32, wherein the flow rate of blood through the cartridge is from about 150 mL/min to about 250 mL/min.

36. The method of any one of claims 1-35, wherein the recipient's blood is passed through the cartridge or contacted with the membrane during the surgical procedure where the organ is transplanted into the subject.

37. The method of any one of claims 1-36, wherein the recipient's blood is passed through the cartridge or contacted with the membrane after a transplant of the organ to the recipient.

38. The method of any one of claims 1-37, wherein the recipient's blood is passed through the cartridge or contacted with the membrane for about 30 minutes to about 72 hours after the transplant of the organ to the recipient.

39. The method of any one of claims 1-38, wherein the recipient's blood is passed through the cartridge or contacted with the membrane for about 1 hour to about 24 hours after the transplant of the organ to the recipient.

40. The method of any one of claims 1-39, wherein the recipient's blood is passed through the cartridge or contacted with the membrane for about 6 hours to about 12 hours after the transplant of the organ to the recipient.

41. The method of any one of claims 1-40, wherein the recipient's blood is passed through the cartridge or contacted with the membrane for about 1 hour, about 3 hours, about 6 hours, about 9 hours, about 12 hours, about 24 hours, about 48 hours or about 72 hours after the transplant of the organ to the recipient.

42. The method of any one of claims 1-41, wherein the recipient's blood is passed through the cartridge or contacted with the membrane within about 12 hours, about 1 day, about 2 days, about 3 days, about 4 days, about 5 days, about 6 days, about 1 week, about 2 weeks, about 3 weeks, about 4 weeks, about 6 weeks, about 8 weeks, about 10 weeks, or about 12 weeks after the transplant of the organ to the recipient.

43. The method of any one of claims 1-42, wherein the recipient's blood is passed through the cartridge or contacted with the membrane within about 0.5-120 hours after the transplant of the organ to the recipient.

44. The method of any one of claims 1-43, wherein the recipient's blood is passed through the cartridge or contacted with the membrane within 30 days prior to the transplant of the organ.

45. The method of any one of claims 1-44, wherein the recipient's blood is passed through the cartridge or contacted with the membrane within 7 days prior to the transplant of the organ.

46. The method of any one of claims 1-45, wherein the cartridge or membrane is connected to the recipient via an extracorporeal circuit comprising a line from an artery or vein of the recipient and a line to a vein of the recipient.

47. The method of any one of claims 1-46, wherein (i) prior to and/or during the transplant, a first cartridge or membrane is connected to the recipient via an extracorporeal circuit comprising a line from an artery or vein of the recipient and a line to a vein of the recipient, and (ii) after the transplant, a second cartridge or membrane is connected to the recipient via an extracorporeal circuit comprising a line from an artery or vein of the recipient and a line to a vein of the recipient.

48. The method of claim 46 or 47, wherein the extracorporeal circuit further comprises one or more of an ultrafiltrate pump, ultrafiltrate pressure sensor, blood sensor, filter pressure sensor, venous pressure sensor, access pressure sensor, IV fluid return pump, ultrafiltration controller, or a temperature regulator.

49. The method of any one of claims 9-48, wherein the ultrafiltration rate of the cartridge is from about 40 mL/min to about 180 mL/min.

50. The method of any one of claims 1-49, wherein the membrane comprises a polymer.

51. The method of any of claims 1-50, wherein the organ is a heart, lung, kidney, liver, intestine, or pancreas.

52. The method of claim 51, wherein the organ is a heart.

53. The method of claim 51, wherein the organ is a lung.

54. The method of claim 51, wherein the organ is a kidney.

55. The method of claim 51, wherein the organ is a liver.

56. The method of claim 51, wherein the organ is an intestine.

57. The method of claim 51, wherein the organ is a pancreas.

58. The method of claims 1-50, wherein the organ is not a liver.

59. The method of claims 1-50, wherein the organ is not a kidney.

60. The method of claims 1-59, wherein the method is not a renal replacement therapy.
